(19) United States
(12) Patent Application Publication
(10) Pub. No.: US 2011/0070241 A1
(43) Pub. Date: Mar. 24, 2011

(54) METHODS FOR MODULATING IMMUNE RESPONSES TO AAV GENE THERAPY VECTORS

(75) Inventor: Yiping Yang, Chapel Hill, NC (US)

(73) Assignee: DUKE UNIVERSITY, Durham, NC (US)

(21) Appl. No.: 12/827,520

(22) Filed: Jun. 30, 2010

Related U.S. Application Data
(60) Provisional application No. 61/269,863, filed on Jun. 30, 2009.

(51) Int. Cl. A61K 39/395; A61K 35/76; A61P 37/06

(52) U.S. Cl. 424/158.1; 424/93.6

(57) ABSTRACT

The present disclosure provides methods of inhibiting an immune response to a viral vector used in gene therapy, such as adeno-associated virus (AAV), which involves co-administration of viral vector and an interfering molecule. The interfering molecule functions by either disrupting the TLR9-MyD88-type I IFN signaling pathway and/or neutralizing Type I IFNs, thereby inhibiting the immune response directed against the viral vector. The methods additionally encompass the step of re-administering the viral vector.

The following disclosure claims priority to U.S. Provisional Application No. 61/269,863 by Yang, Y. and entitled “Methods for Modulating Immune Responses to AAV Gene Therapy Vectors,” filed Jun. 30, 2009, the contents of which are herein incorporated by reference.

### FEDERAL FUNDING LEGEND

This disclosure was produced in part using finds from the Federal Government under NIH grant nos. CA111807 and CA047741. Accordingly, the Federal government has certain rights in this disclosure.

### FIELD OF THE INVENTION

The present disclosure relates generally to fields of immunology and gene therapy. Specifically, the present disclosure relates to novel methods of modulating immune responses to viral (e.g., adeno-associated virus (AAV))-gene therapy vectors.

### BACKGROUND OF THE INVENTION

Adeno-associated virus (AAV) is a non-enveloped, single-stranded DNA virus with a genome of ˜5 kb. It is a member of Parvoviridae family and requires a helper virus such as adenovirus or herpes simplex virus for replication. Despite a limited packaging capacity (<4.7 kb), AAV has many attractive features for use as a vector for in vivo gene therapy, including the ability to transduce a variety of cells, low immunogenicity and toxicity, and the ability to establish long-term expression of the transgene in viva (Wu, Z. et al. 2006 Mol. Ther. 14:316-327). So far, AAV vectors have been used in preclinical and clinical studies for a variety of diseases, including hemophilia (Snyder, R. O. et al. 1999 Nat. Med. 5:64-70; Herzog, R. W. et al. 1999 Nat. Med. 5:56-63; Kay, M. A. et al. 2000 Nat. Genet. 24:257-261; Marano, C. S. et al. 2006 Nat. Med. 12:342-347), Duchenne muscular dystrophy (Greelish, J. P. et al. 1999 Nat. Med. 5:439-443; Gregorevic, P. et al. 2006 Nat. Med. 12:787-789), α1-antitrypsin deficiency (Song, S. et al. 1998 Proc. Natl. Acad. Sci. USA 95:14384-14388; Stedman, H. et al. 2000 Hum. Gene Ther. 11:777-790), and cystic fibrosis (Flotte, T. R. et al. 1993 Proc. Natl. Acad. Sci. USA 90:10613-10617; Wagner, J. A. et al. 2002 Hum Gene Ther. 13:1349-1359). Among 9 serotypes of AAV that have been developed for gene therapy, serotype 2 (AAV2) is the most extensively studied (Wu, Z. et al. 2006 Mol. Ther. 14:316-327).

The ability of AAV vectors to achieve long-term expression of the transgene product has been attributed to their relatively low immunogenicity (Fisher, K. J. et al. 1997 Nat. Med. 3:306-312; Herzog, R. W. et al. 1997 Proc. Natl. Acad. Sci. USA 94:5804-5809; Wang, L. et al. 1999 Proc. Natl. Acad. Sci USA 96:3906-3910). However, in some experimental settings, attendant immune responses have compromised the outcome of AAV-mediated gene therapy. In fact, for this reason, AAV vectors have been developed as a vaccine vehicle for infectious diseases and cancer (Manning, W. C. et al. 1997 J. Virol. 71:7960-7962; Liu, D. W. et al. 2000 J. Virol. 74:2888-2894; Xin, K. Q. et al. 2001 Hunt. Gene Ther. 12:1047-1061). Several factors may influence the occurrence of immune responses to AAV, including the vector dose and serotype, the nature of the transgene, the route of administration, pre-existing immunity to AAV, and the host species (Vandenberghe, L. H. et al. 2007 Cum Gene Ther. 7:325-333). It has been suggested that activation of transgene-specific T cell response is due to cross-presentation of phagocytosed transgene-derived antigens in the context of MHC class I by dendritic cells (DCs) (Manning, W. C. et al. 1997 J. Virol. 71:7960-7962; Sarukhan, A. et al. 2007 J. Virol. 75:269-277). In addition, cross-presentation of the input vector capsid proteins by DCs can activate capsid-specific T cell response (Vandenberghe, L. H. et al. 2006 Nat. Med. 12:967-971; Wang, Z. et al. 2007 Hum Gene Ther. 18:18-26; Wang, L. et al 2007 Hum. Gene Ther. 18:185-194; Li, C. et al. 2007 J. Virol. 81:7540-7547). Furthermore, efficient activation of B cell response by AAV vectors leads to production of neutralizing antibodies against viral capsids, which limit effective re-administration of the vector (Chirmule, N. et al. 2000 J. Virol. 74:2420-2425; Peden, C. S. et al. 2004 J. Virol. 78:6344-6359; Scallan, C. D. et al. 2006 Blood 107:1810-1817). Collectively, these observations suggest that AAV vectors are not intrinsically inert in eliciting host immune responses.

The concern for immune responses to AAV vectors has been substantiated by the outcome of a recent clinical trial in hemophilia B patients (Manno, C. S. et al. 2006 Nat. Med. 12:342-347). In this trial, hepatic delivery of AAV2 vectors encoding factor IX led to therapeutic levels of transgene-encoded factor IX in one patient. However, the therapeutic levels of factor IX were only transient. The gradual decline in factor IX was accompanied by a transient transaminitis and the detection of AAV capsid-specific T cells. Overall, the patient's clinical course was compatible with immune-mediated destruction of AAV-transduced hepatocytes. Taken together, the above observations in mice and humans suggest that adaptive immune responses to AAV vectors have posed a major challenge in AAV-mediated gene therapy in vivo.

Critical for the development of effective strategies to circumvent these hurdles is to understand what controls the induction of adaptive immunity to AAV. Recent advances in immunology have suggested a crucial role for the innate immune system in promoting adaptive immune responses (Iwasaki, A. et al. 2004 Nat. Immunol. 5:987-995; Pulendran, B. et al. 2006 Cell 124:849-863). The phylogenetically conserved innate immune system represents the first line of defense against invading pathogens through recognition of pathogen-associated molecular patterns (PAMPs) by a set of receptors called pattern recognition receptors (PRRs) (Akira, S. et al. 2006 Cell 124:783-801). The best-studied family of PRRs is the Toll-like receptors (TLRs) that are expressed on various innate immune cells such as DCs and macrophages. Upon recognition of PAMPs, TLRs trigger a series of signaling cascades leading to induction of anti-microbial genes and inflammatory cytokines, which results in direct killing of the invading pathogens as well as promoting the initiation of adaptive immune responses (Iwasaki, A. et al. 2004 Nat. Immunol. 5:987-995).

How AAV activates the innate immune system remains unknown. In this study, utilizing DCs deficient for genes involved in the TLR pathways, we showed that AAV2 activated plasmacytoid DCs (pDCs) to produce type I interferons (IFNs). The innate immune recognition of AAV by pDCs was mediated by TLR9 and dependent on MyD88. Activation of the TLR9-MyD88 pathway was independent of the nature of the transgene. Similarly, other serotypes of AAV such as AAV1 and AAV9 also activated innate immunity through the TLR9-MyD88 pathway. In vivo, the TLR9-MyD88 pathway was critical for the activation of CD8 T cell responses to both the transgene product and the AAV capsid, leading to the loss of transgene expression, and the formation of anti-transgene antibody and neutralizing antibodies to AAV vectors. This was mediated by TLR9-induced production of type I IFNs. We further demonstrated that AAV vectors also activated human pDCs to induce type I IFNs via TLR9. Collectively, these observations suggest that strategies to block the TLR9-MyD88-type I IFN pathway may improve the clinical outcome of AAV-mediated gene therapy.

### SUMMARY OF THE DISCLOSURE

The present disclosure provides methods for modulating immune responses to viral gene therapy vectors (e.g., AAV gene therapy vectors), thereby resulting in modulated immune responses to the viral vector to accomplish the therapy.

One aspect of the present disclosure provides a method of inhibiting in a subject formation of neutralizing antibodies directed against a viral vector, such as AAV, comprising, consisting of, or consisting essentially of co-administering to the subject the adenovirus and an interfering molecule, wherein the interfering molecule is capable of disrupting the TLR9-MyD88-type I IFN signaling pathway.

Another aspect of the present disclosure provides a method of inhibiting in a subject formation of an immune response directed against a viral vector, such as AAV, comprising, consisting of, or consisting essentially of co-administering to the subject the adenovirus and an interfering molecule directed against type I interferons, wherein the formation of the immune response is inhibited.

In one embodiment, the method comprising the step of re-administering the adenovirus to the subject. In other embodiments, the interfering molecule is administered simultaneously with the adenovirus. In yet another embodiment, the interfering molecule is administered prior to the administration of said adenovirus. In other embodiments, the interfering molecule is administered subsequently after the administration of the adenovirus.

In another embodiment, the interfering molecule is selected from the group consisting of an antagonist, antisense RNA, siRNA, aptamers, and combinations thereof. In certain embodiments, the interfering molecule comprises an antagonist. In other embodiments, the antagonist comprises H154ODN. In yet another embodiment, the antagonist comprises ODN2088.

In another embodiment, the interfering molecule comprises a polyclonal neutralizing antibody directed to INF-α or IFN-β.

These and other novel features and advantages of the disclosure will be fully understood from the following detailed description and the accompanying drawings.

### FIGURES AND DRAWINGS

FIGS. 1A-1D show how AAV2 mainly stimulates bone marrow derived pDCs to secrete type I IFNs. pDCs and cDCs were generated from bone marrow cells in the presence of Flt-3 ligand and GM-CSF, respectively, and purified by FACS sorting. Cells (1×10⁶) were then stimulated with AAV2-lacZ (2×10¹⁰ vg), Ad-lacZ (MOI of 250) or left unstimulated (medium), for 18 Ms and the supernatants were assayed for the secretion of IFN-α (FIG. 1A), IFN-β (FIG. 1B), IL-6 (FIG. 1C), and TNF-α (FIG. 1D) by ELISA. Representative data of 3 independent experiments are shown.

FIGS. 2A and 2B show how AAV2 activates endogenous pDCs, but not non-pDCs, to produce type I IFNs. 2.5×10⁵ of splenic pDCs, CDCs, hepatic Kupffer cells (KC), or peritoneal macrophages (Mø) were either unstimulated (Medium) or stimulated with AAV2-lacZ (5×10⁹ vg) or Ad-lacZ (moil of 250) for 18 hr. The culture supernatants were assayed for the secretion of IFN-α (FIG. 2A) and IL-6 (FIG. 2B). Representative data of 3 independent experiments are shown.

FIGS. 3A-3D show that pDC recognition of AAV2 is mediated by TLR9 and dependent on MyD88. (FIGS. 3A-B) pDCs (1×10⁶) generated from bone marrow cells of wild type (WT), MyD88−/−, or TRIF−/− C57BL/6 mice were purified and stimulated with AAV2-lacZ (2×10¹⁰ vg) for 18 hr and the supernatants were assayed for the secretion of IFN-α (FIG. 3A) and IFN-β (FIG. 3B) by ELISA. (FIGS. 3C-D) pDCs generated from bone marrow cells of WT, TLR2−/−, or TLR9−/− C57BL16 mice were stimulated with AAV2-lacZ for 18 his and the supernatants were assayed for the secretion of IFN-α (FIG. 3C) and IFN-β (FIG. 3D) by ELISA. Representative data of 3 independent experiments are shown.

FIGS. 4A and 4B show how DNase I treatment does not affect the ability of AAV to stimulate pDCs. pDCs (1×10⁶) generated from bone marrow cells were purified and stimulated with AAV2-lacZ (2×10¹⁰ vg) or DNase I-treated (30 min at 37° C.) AAV-lacZ for 18 hr and the supernatants were assayed for the secretion of IFN-α (FIG. 4A) and IFN-β (FIG. 4B) by ELISA. Representative data of 2 independent experiments are shown.

FIGS. 5A-5E show how the activation of the TLR9-MyD88 pathway by AAV is independent of the nature of the transgene or AAV serotypes. 1×10⁶ of pDCs generated from WT, MyD88−/−, or TLR9−/− mice were stimulated with 2×10¹⁰ vg of AAV2-lacZ (FIG. 5A), AAV2-HA (FIG. 5B), AAV2-GFP (FIG. 5C), AAV1-GFP (FIG. 5D), or AAV9-GFP (FIG. 5E) for 18 hr and the supernatants were assayed for the secretion of IFN-β by ELISA. Representative data of 3 independent experiments are shown.

FIGS. 6A-6F show how the lack of TLR9-MyD88 signaling diminishes CD8 T cell responses to the AAV capsid and the transgene product, and prolongs the transgene expression. AAV2-HA (1×10¹¹ vg) was injected intramuscularly into WT, TLR9−/−, or MyD88−/− BALB/c mice. (FIG. 6A) 12, 26 and 60 days later, the infected muscles were harvested and analyzed for HA expression by immunohistochemistry. (FIG. 6B) CD5+ T cells purified from splenocytes at day 26 after infection, along with uninfected WT splenocytes (Control) were restimulated with AAV2-HA at 0, 50, 500, or 5000 vg/cell. Proliferation of AAV-specific T cells was analyzed by ³H-thymidine incorporation. Data reflect the mean±s.d. of stimulation index, calculated by dividing ³H counts in cpm in the presence of viral stimulation by those in the absence of stimulation, as a function of different virus doses. (FIGS. 6C-F) At days 12 and 26 after infection, splenocytes were harvested and stimulated with either AAV2 capsid epitope peptide (FIGS. 6C, D) or HA epitope peptide (FIGS. 6E, F) for 5 hr and assayed for intracellular IFN-γ secretion by CD8 T cells. The FACS plots show percentages of IFN-γ-producing CD8 T cells among total CD8 T cells (FIG. 6C, E). The mean percentages ±s.d. of IFN-γ-producing CD8 T cells among total CD8 T cells are also shown (FIG. 6D, F). Representative results of 3 independent experiments are shown.

FIGS. 7A-7E show how the formation of anti-transgene and AAV-neutralizing antibodies is also dependent on the TLR9-MyD88 pathway. WT, TLR9−/−, or MyD88−/− mice were injected with AAV2-HA intramuscularly. (FIGS. 7A-B) Serum samples were harvested at day 36 for the measurement of anti-HA antibody titer by ELISA (FIG. 7A) as well as neutralizing antibody titers to AAV vectors (FIG. 7B). (FIGS. 7C-E) Sera were also analyzed for vector-specific IgG2a (FIGS. 7C), IgG1 (FIG. 7D), and IgG3 (FIG. 7E) by ELISA. Data reflect the mean±s.d. of reciprocal endpoint titers. Data shown are representative of 3 independent experiments.

FIGS. 8A-8D show how type I IFNs play a critical role in adaptive immune responses to AAV. AAV2-HA was injected intramuscularly into WT or IFNR−/− mice. 12 and 36 days later, the infected muscles were harvested and analyzed for HA expression by immunohistochemistry (FIG. 8A). CD5+ T cells purified from splenocytes at day 36 after infection, along with uninfected WT splenocytes (Naive) were restimulated with AAV2-HA at 0, 50, 500, or 5000 vg/cell. Proliferation of AAV-specific T cells was analyzed by ³H-thymidine incorporation (FIG. 8B). Data reflect the mean±s.d. of stimulation index, calculated by dividing ³H counts in cpm in the presence of viral stimulation by those in the absence of stimulation, as a function of different virus doses. (FIG. 8C-D) Serum samples were harvested at day 36 for the measurement of anti-HA (FIG. 8C) and AAV-neutralizing (FIG. 8D) antibody titers. Data shown are representative of 2 independent experiments.

FIGS. 9A-9B show how activation of human pDCs by AAV is also mediated by TLR9. (FIG. 9A) 1×10⁵ of human pDCs or monocytes were purified from PBMCs, and stimulated with either AAV2-lacZ (2×10⁹ vg) or left unstimulated (Medium) for 18 hr. Cells were then harvested, and total RNA was treated with DNase I and assayed for the expression of human IFN-α (hIFN-α) and IFNI-β (hIFN-β) by RT-PCR. (FIG. 9B) Human pDCs (1×10⁵) were either unstimulated (Medium), or stimulated with AAV2-lacZ (2×10⁹ vg) or a TLR9 agonist, CpG-A ODN (5 μg/ml). In some experiments, cells were pre-treated with a TLR9 antagonist, H154 ODN (10 μM) for 30 min, followed by the stimulation with AAV2-lacZ or CpG-A. 18 hr later, cellular RNA was analyzed for the induction of hIFN-α and hIFN-β by semi-quantitative RT-PCR using 5 fold serial dilution of the template. Human ribosomal protein 514 was used as an internal loading control. Data shown are representative of 2 independent experiments.

FIGS. 10A-10B show the kinetics of cytokine production by pDCs upon AAV2 infection. pDCs were generated from bone marrow cells in the presence of Flt-3 ligand and purified by FACS. In FIG. 10A, cells (1×10⁶) were stimulated with AAV2-LacZ at indicated doses for 18 hr and the supernatants were assayed for IFN-α and IL-6 secretion by ELISA. In FIG. 10B, cells were stimulated with AAV2-lacZ at 2×10¹⁰ vg for 0, 6, 12, 18, or 48 h, and the supernatants were measured for IFN-α and IL-6 by ELISA.

FIG. 11 shows that AAV promotes DC maturation via TLR9. pDCs were generated from bone marrow cells in the presence of Flt-3 and purified by FACS sorting. Cells (1×10⁶) were then stimulated with AAV2-lacZ (2×10¹⁰ vg), CpG (5 μg/ml), or left unstimulated (medium alone) for 18 hr and analyzed for expression of CD86 by FACS. The mean fluorescence intensity is indicated.

FIG. 12 shows the infiltration of CD8 T cells into AAV-infected muscles. AAV2-HA (1×10¹¹ vg) was injected intramuscularly into WT, TLR9−/−, or MyD88−/− mice. After 26 days, the infected muscles were harvested and analyzed for CD8 T cell infiltration by immunohistochemistry.

FIG. 13 is a schematic showing the TLR9-MyD88 signaling pathway in pDC cells.

FIG. 14 is a graph showing that the addition of TLR9 antagonist inhibits type I IFN production by pDCs upon AAV infection. Purified pDCs were stimulated with AAV2-lacZ (2×10¹⁰ vg) or a TLR9 agonist CpG ODN (5 μg/ml) in the presence of 0, 5 or 50 μM of a TLR9 antagonist, ODN2088 for 18 hrs and the supernatants were assayed for the secretion of IFN-α by ELISA.

FIGS. 15A-C show that Type I IFN blockade diminishes adaptive immune responses to AAV. AAV2-HA was injected intramuscularly into BALB/c mice that had been treated with neutralizing antibodies to IFN-α and IFN-β (IFN-αβ Ab) or control antibody, (Control Ab). In FIG. 15A, 12 and 26 days post treatment, the infected muscles were harvested and analyzed for HA expression by immunohistochemistry. In FIG. 15B, CD5⁺ T cells purified from splenocytes at day 26 after injection, along with uninfected WT splenocytes (Naïve) were restimulated with AAV2-HA at 0, 50, 500 or 5000 vg/cell. Proliferation of AAV-specific T cells were analyzed by ³H-Thymidine incorporation. Data reflect the mean±s.d. of stimulation index, calculated by dividing ³H counts in cpm in the presence of viral stimulation by those in the absence of stimulation, as a function of different virus doses. In FIG. 15C, serum samples were harvested at day 26 for the measurement of AAV-neutralizing antibody titers.

### DESCRIPTION OF EMBODIMENTS

Unless otherwise defined, all technical terms used herein have the same meaning as commonly understood by one of ordinary skill in the art to which this disclosure belongs.

The articles “a” and “an” are used herein to refer to one or to more than one (i.e. at least one) of the grammatical object of the article. By way of example, “an element” means at least one element and can include more than one element.

### DEFINITIONS

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, mouse, rat, horse, cow, chickens, amphibians, reptiles, and the like. Preferably, the subject is a mammal. More preferably, the subject is a human patient.

As used herein, the term “type I interferon (IFN)” refers to those molecules that are capable of binding the IFN-α/β receptor complex. Such interferons include IFN-α, IFN-β, IFN-κ, IFN-ε, IFN-ε, IFN-τ and IFN-ω.

The term “administering” or “administered” as used herein is meant to include both parenteral and/or oral administration, all of which are described in more detail in the “pharmaceutical compositions” section below. By “parenteral” is meant intravenous, subcutaneous or intramuscular administration. In the methods of the subject disclosure, the interfering molecules of the present disclosure may be administered alone, simultaneously with one or more other interfering molecule, or the compounds may be administered sequentially, in either order. It will be appreciated that the actual preferred method and order of administration will vary according to, inter alia, the particular preparation of interfering molecules being utilized, the particular formulation(s) of the one or more other interfering molecules being utilized. The optimal method and order of administration of the compounds of the disclosure for a given set of conditions can be ascertained by those skilled in the art using conventional techniques and in view of the information set out herein. The term “administering” or “administered” also refers to oral sublingual, buccal, transnasal, transdermal, rectal, intramascular, intravenous, intraventricular, intrathecal, and subcutaneous routes. In accordance with good clinical practice, it is preferred to administer the instant compounds at a concentration level which will produce effective beneficial effects without causing any harmful or untoward side effects.

As used herein, the term “attenuated immunostimulatory properties” means a decreased, lesser or suppressed immune response by the interfering compound of the present disclosure as compared to an appropriate control.

The terms “suppress”, “inhibit”, “block”, “decrease”, “attenuate,” “downregulated” “reduce” or the like, denote quantitative differences between two states, refer to at least statistically significant differences between the two states. For example, “an amount effective to inhibit a CD8 T cell response” means that the CD8 T cell immune response in a subject treated with a viral vector and interfering molecule will be at least statistically significantly different from the a subject treated with a viral vector alone. Such terms are applied herein to, for example cytokine production, CD8 T cell activation and the like.

The term “immune response” includes any response associated with immunity including, but not limited to, increases or decreases in cytokine expression, production or secretion (e.g., IL-12, IL-10, TGF-β or TNF-α expression, production or secretion), cytotoxicity, immune cell migration, antibody production and/or immune cellular responses. The phrase “modulating an immune response” or “modulation of an immune response” includes downregulation, inhibition or decreasing an immune response as defined herein. For example, an immune response can be downregulated, suppressed, or blocked by use of an interfering molecule of the present disclosure (e.g., a neutralizing antibody).

As used herein, the phrase “gene therapy” refers to the transfer of genetic material (e.g., DNA or RNA) of interest into a host to treat or prevent a genetic or acquired disease or condition. The genetic material of interest encodes a product (e.g., a protein polypeptide, peptide or functional RNA) whose production in vivo is desired. For example, the genetic material of interest can encode a hormone, receptor, enzyme or (poly) peptide of therapeutic value. Examples of genetic material of interest include DNA encoding: the cystic fibrosis transmembrane regulator (CFTR), Factor VIII, Factor IX, low density lipoprotein receptor, β-galactosidase, α-galactosidase, β-glucocerebrosidase, insulin, parathyroid hormone, and α-1-antitrypsin.

Suitable viral vectors useful in gene therapy are well known, including retroviruses, vaccinia viruses, poxviruses, adenoviruses, and adeno-associated viruses (AAV), among others. Such viral vectors may also be recombinant. The methods described in the present disclosure are anticipated to be useful with any virus which forms the basis of a gene therapy vector. However, exemplary viral vectors for use in the methods described herein are adenovirus and adeno-associated virus (AAV). As used herein, the terms “viral vector” and “recombinant viral vector” are used interchangeably herein and refer to any of the well known virus vectors used in gene therapy. Similarly, the terms “adenovirus” and “recombinant adenovirus” are used interchangeably herein and refer to any of the known adenovirus used in gene therapy, such as human type C adenovirus, including serotypes Ad2 and Ad5, which have been rendered replication defective for gene therapy by deleting the early gene locus that encodes E1a and E1b (see, e.g., Kozarsky, K. F. and Wilson, J. M., (1993) Curr. Opin. Genet. Dev., 3:499-503). Further, the term “AAV” refers to all adeno-associated viruses that are used in gene therapy, including all associated serotypes, such as AAV2.

The selection of the virus for the recombinant vectors useful in the methods described herein, including the viral type, e.g., AAV, and strain are not anticipated to limit the following disclosure.

Similarly, selection of the gene of interest contained within the viral vector is not a limitation to the present disclosure. As used herein, the term “gene of interest” refers to the gene that is included in the viral vector thereby treating the disease suffered by the subject. In certain embodiments, the viral vector is used as a vaccine. In certain embodiments, the viral vector contains a DNA sequence of interest that encodes a protein or a peptide. Upon administering of such a vector to a subject, the protein or peptide encoded by the DNA sequence of interest is expressed and stimulates an immune response specific to the protein or peptide encoded by the DNA sequence of interest.

The methods described herein are anticipated to be useful with any gene of interest, for any particular disease. As used herein, the term “disease” refers to an abnormal condition of a subject that often impairs bodily functions. More broadly, the term “disease” refers to any condition that causes discomfort, dysfunction, distress, social problems, and/or death to the subject resulting from genetic abnormalities, cancer and/or infection with pathogenic organisms. The term “disease” and “illness” can be used interchangeably. Suitable genes of interest for delivery to a patient in a viral vector for gene therapy are known to those skilled in the art. These therapeutic nucleic acid sequences typically encode products for administration and expression in a subject in vivo or ex vivo to replace or correct an inherited or non-inherited genetic defect or treat an epigenetic disorder or disease. Such therapeutic genes include, but are not limited to, Factor IX for the treatment of hemophilia, DMD Becker allele for the treatment of Duchenne muscular dystrophy, genes related to the treatment of α-1-antitrypsin deficiency, the cystic fibrosis transmembrane regulator gene (CFTR) for the treatment of cystic fibrosis, and a number of genes which may be readily selected by one of skill in the art. Thus, the selection of the gene of interest is not considered to be a limitation of this disclosure, as such the selection is within the knowledge of those skilled in the art.

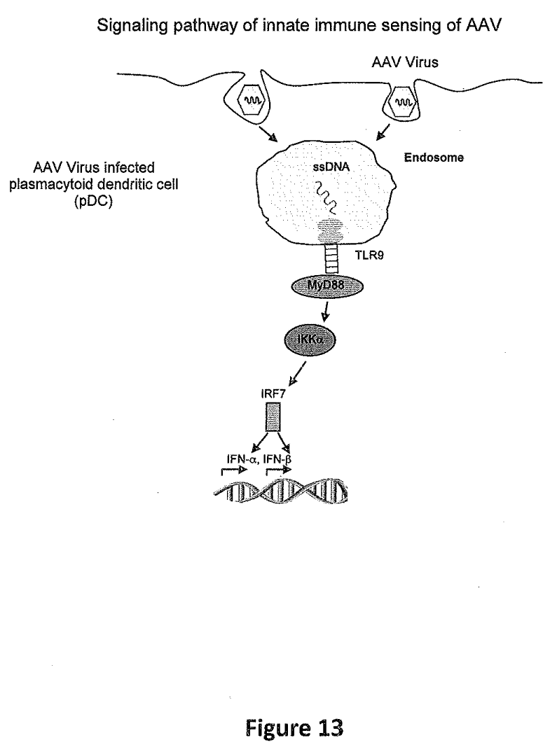

According to the present disclosure, it has been discovered that the innate immune recognition of AAV is through the TLR9-MyD88 pathway in plasmacytoid DCs (pDCs), which leads to the production of type I interferons (IFNs) (FIG. 13). The TLR9-MyD88-type I IFN pathway is critical for the activation of CD8 T cell responses to both transgene product and the AAV vector, leading to the loss of transgene expression. Therefore, strategies targeted to interfere with the TLR9-MyD88-type I IFN signaling pathway in pDCs will minimize T cell responses to viral capsid antigen and the transgene product, thus lead to prolonged transgene expression and reduction in inflammation, improving the safety and efficacy of AAV vectors for gene therapy in humans. In addition, blockade of TLR9-MyD88-type I IFN signaling pathway will also diminish antibody responses to the transgene product as well as neutralizing antibody to AAV vector. Reduction of antibody to transgene product will prevent neutralization of secreted transgene-derived product such as in the case of factor IX, whereas reduction of antibody to AAV vectors will allow for re-administration of the vector if needed.

As used herein, the term “interfering molecule” refers to any molecule that is capable of modulating an immune response directed against an adenoviral vector. In certain embodiments, the “interfering molecule” is capable of disrupting the TLR9-MyD88 signaling pathway. In other embodiments, the interfering molecule is capable if disrupting Type I IFNs generated as a result of introduction of an adenoviral vector into a subject. Examples of suitable interfering molecules include, but are not limited to, small molecules, antibodies, antisense RNAs, siRNAs, cDNAs, dominant-negative forms of molecules such as TLR9, peptides, neutralizing antibodies, combinations thereof, and the like.

According to one embodiment of the present disclosure, antagonists specific to TLR9, such as H154 ODN and ODN2088, are used to block the TLR9-MyD88 pathway. As the activation of pDCs depends on an intact TLR9-MyD88 pathway, such blocking will inhibit/block the innate sensing of viruses by pDCs.

In yet another embodiment of the present disclosure, antibodies of type I IFNs capable of neutralizing, for example, IFN-α and IFN-β may be administered to the subject. Since type I IFNs are required for adaptive immune responses to viral vectors, such as AAV, this approach will lead to stable transgene expression as well as diminished neutralizing antibodies to both transgene product and the viral vector.

In yet other embodiments, antisense RNA and/or siRNA specifically targeting type I IFNs in pDCs are used to block the production of type I IFNs, thereby achieving stable transgene expression and diminished antibody responses to transgene product and viral vector, such as AAV; and methods to manipulate viral vector trafficking to the endosomes of pDCs where recognition of the viral vector, such as AAV, by TLR9 takes place will achieve the same goal.

According to the present disclosure, a “therapeutically effective amount” of an interfering molecule is an amount which is sufficient for the desired pharmacological effect. A suitable amount or dosage of the interfering molecule will depend primarily on the amount of the viral vector bearing the gene of interest which is initially administered to the subject and the type of interfering molecule selected. Other secondary factors such as the condition being treated, the age, weight, general health, and immune status of the subject, may be considered by a physician in determining the dosage of interfering molecule to be delivered to the subject. Various dosages may be determined by one of skill in the art to balance the therapeutic benefit against any side effects.

In another aspect, the present disclosure provides methods for treating a disease in a subject by in vivo viral vector gene therapy comprising, consisting of, or consisting essentially of administering to the subject a therapeutically effective amount of a viral vector, such as AAV, comprising a gene of interest and a therapeutically effective amount of an interfering molecule, wherein the interfering molecule is capable of disrupting the TLR9-MyD88-type I IFN signaling pathway.

In another aspect, the present disclosure provides methods for treating a disease in a subject by in vivo viral vector gene therapy comprising, consisting of, or consisting essentially of administering to the subject a therapeutically effective amount of a viral vector, such as AAV, comprising a gene of interest and a therapeutically effective amount of an interfering molecule, wherein the interfering molecule is capable of neutralizing type I IFNs.

Another aspect of the present disclosure provides a method of enhancing the efficacy of viral vector gene therapy treatment in a subject comprising, consisting of, or consisting essentially of administering to the subject a viral vector, such as AAV, comprising a gene of interest and an interfering molecule.

Method of the Disclosure

The viral vector bearing a gene of interest may be administered to a subject and is preferably suspended in a biologically compatible solution or pharmaceutically acceptable delivery vehicle. A suitable vehicle includes sterile saline. Other aqueous and non-aqueous isotonic sterile injection solutions and aqueous and non-aqueous sterile suspensions known to be pharmaceutically acceptable carriers and well known to those of skill in the art may be utilized for this purpose.

The viral vector is administered in sufficient amounts to transfect the desired cells and provide sufficient levels of transduction and expression of the gene of interest. It is to provide a therapeutic benefit without undue adverse or with medically acceptable physiological effects which can be determined by those skilled in the medical arts. Conventional and pharmaceutically acceptable routes of administration include direct delivery to the target organ, tissue or site, intranasal, intravenous, intramuscular, subcutaneous, intradermal, oral and other parental routes of administration. If desired, the above mentioned routes of administration may be combined.

Dosages of the viral vector will depend primarily on factors such as the condition being treated, the selected gene, the age, weight and health of the patient, and may thus vary among subjects. For instance, a therapeutically effective human dosage of the viral vectors is generally in the range of from about 20 to about 50 ml of saline solution containing concentrations of from about 1×10⁷ to 1×10¹⁰ pfu/ml viruses. A preferred adult human dosage is about 20 ml saline solution at the above concentrations. The dosage will be adjusted to balance the therapeutic benefit against any side effects. The levels of expression of the gene of interest can be monitored to determine the selection, adjustment or frequency of dosage administration.

The method of this disclosure involves the co-administration of the selected interfering molecule(s) with the selected recombinant viral vector. The co-administration occurs so that the interfering molecule and vector are administered within a close time proximity to each other. It is presently preferred to administer the interfering molecule concurrently with or no longer than one day prior to the administration of the vector. The interfering molecule may be administered separately from the recombinant vector, or, if desired, it may be administered in admixture with the recombinant vector.

For example, where a TLR9 antagonist such as H154ODN or ODN2088 is the interfering molecule, the interfering molecule is desirably administered in close time proximity to the administration of the viral vector used for gene therapy. Alternatively, a TLR9 antagonist may administered essentially simultaneously with the viral vector. In other embodiments, such as those cases where a neutralizing antibody is administered, the interfering molecule may be administered shortly after (e.g., 1 day, 2 days, 3, days, 4 days, 5 days) the administration of the viral vector used for gene therapy.

The interfering molecule may be administered in a pharmaceutically acceptable carrier or diluent, such as saline. For example, when formulated separately from the viral vector, the interfering molecule is desirably suspended in saline solution. Such a solution may contain conventional components, e.g. pH adjusters, preservatives and the like. Such components are known and may be readily selected by one of skill in the art.

Alternatively, the interfering molecule may be itself administered as DNA, either separately from the vector or admixed with the recombinant vector bearing the gene of interest. Methods exist in the art for the pharmaceutical preparation of the interfering molecule as protein or as DNA (see, e.g., J. Cohen et al. (1993) Science 259:1691-1692 regarding DNA vaccines) Desirably the interfering molecule is administered by the same route as the recombinant vector.

The interfering molecule may be formulated directly into the composition containing the viral vector administered to the subject. Alternatively, the interfering molecule may be administered separately, preferably shortly before or after administration of the viral vector. In another alternative, a composition containing one interfering molecule may be administered separately from a composition containing a second interfering molecule, and so on depending on the number of interfering molecules administered. These administrations may independently be before, simultaneously with, or after administration of the viral vector.

The administration of the selected interfering molecule may be repeated during the treatment with the recombinant adenovirus vector carrying the gene of interest, during the period of time that the gene of interest is expressed, as monitored by assays suitable to the gene of interest or its intended effect) or with every booster of the recombinant vector. Alternatively, each reinjection of the same viral vector may employ a different interfering molecule.

One advantage of the method of this disclosure is that it represents a transient manipulation necessary only at the time of administration of the gene therapy vector. It is also anticipated to be safer than strategies based on induction of tolerance which may permanently impair the ability of the recipient to respond to adenovirus infections. Furthermore, the use of interfering molecules such as the antagonists or neutralizing antibodies in preference to agents such as cyclosporin or cyclophosphamide is predicted to be safer than generalized immune suppression because the transient immune modulation is selective.

### EXAMPLES

### Examples 1-12

### TLR9-MyD88 Pathway is Critical for Adaptive Immune Responses to AAV Gene Therapy Vectors

### Example 1

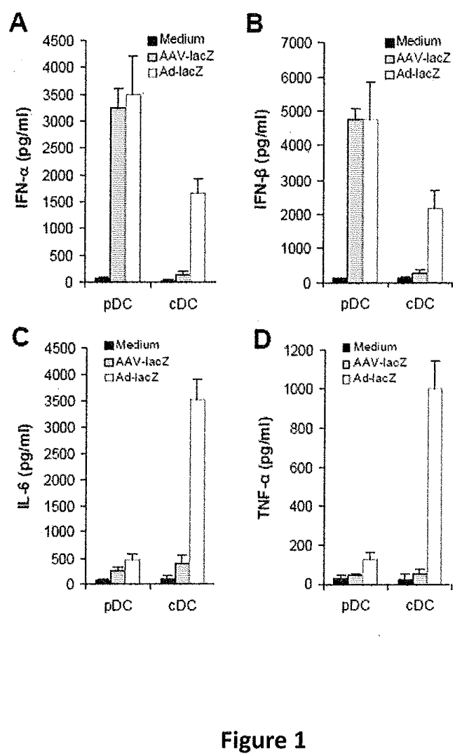

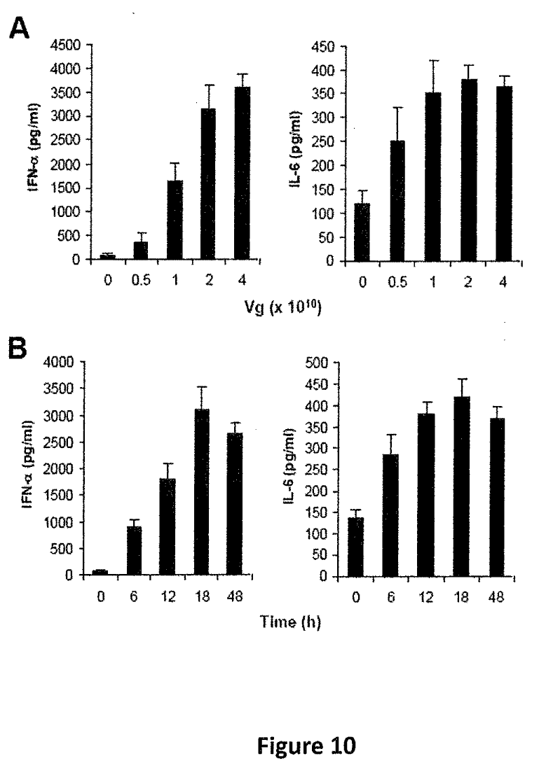

AAV2 activates pDCs to produce type I IFNs. Studies have shown that both pDCs and conventional DCs (cDCs) play a pivotal role in innate immune sensing of viruses (Kawai, T. et al. 2006 Nat. Immunol. 7:131-137). Indeed, we have demonstrated that the innate immune recognition of adenoviral vectors by pDCs is mediated by TLR9, whereas that by non-pDCs such as cDCs and macrophages is TLR-independent (Zhu, J. et al. 2007 J. Virol. 81:3170-3180). We thus utilized both pDCs and CDCs to study innate immune response to AAV. pDCs and cDCs were generated from bone marrow cells in the presence of Flt-3 ligand and GM-CSF, respectively, as we previously described (Zhu, J. et al. 2007 J. Virol. 81:3170-3180). pDCs and CDCs, identified as CD11c+B220+mPDCA-1+ and CD11c+B220-mPDCA-1−, respectively, were then purified by FACS sorting and stimulated with recombinant AAV2 encoding lacZ (AAV2-lacZ, 2×1010 vg) or E1-deleted adenovirus encoding lacZ (Ad-lacZ, MOI of 250) for 18 h, and the culture supernatants were assayed for the secretion of type I IFNs such as IFN-α and IFN-β, and pro-inflammatory cytokines such as IL-6 and TNF-α. Similar to those infected with Ad-lacZ, pDCs stimulated with AAV2-lacZ produced high levels of IFN-α (FIG. 1A) and IFN-(FIG. 1B), but very low levels of IL-6 (FIG. 1C) and TNF-α (FIG. 1D). However, little or no type I IFNs, or pro-inflammatory cytokines were produced by cDCs upon AAV2-lacZ infection (FIG. 1). This was in striking contrast to cDCs stimulated with Ad-lacZ, which produced high levels of IL-6 (FIG. 1C) and TNF-a (FIG. 1D) as well as IFN-α (FIG. 1A) and IFN-β (FIG. 1B). The dose and the time point chosen for AAV2-lacZ in these studies were based on our pilot experiments that optimal responses were obtained with DCs stimulated for 18 h at 2×10¹⁰ vg (FIG. 10), and the dosing for Ad-lacZ was based on our published data (Zhu, J. et al. 2007 J. Virol. 81:3170-3180). Collectively, these results indicate that AAV2-lacZ mainly activates pDCs to produce type I IFNs.

### Example 2

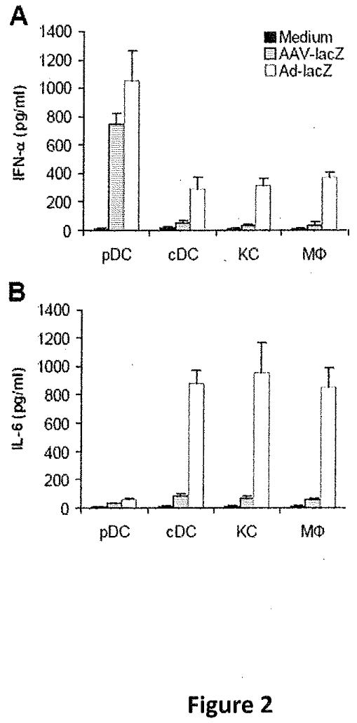

AAV primarily activates pDC, but not non pDCs, to produce type I IFNs. We next examined whether endogenous pDCs and cDCs behaved similarly in response to AAV2-lacZ infection. Splenic pDCs and cDCs were purified by FACS sorting, and the purified DCs were stimulated with AAV2-lacZ or Ad-lacZ and measured for secretion of IFN-γ and IL-6. Again, similar to Ad-lacZ, AAV2-lacZ stimulated endogenous pDCs, but not cDCs, to secrete IFN-γ (FIG. 2A). In contrast to adenoviral infection, no significant levels of IL-6 were produced by endogenous cDCs upon AAV infection (FIG. 2B). We also investigated how other non-pDCs such as macrophages and hepatic Kupffer cells responded to AAV infection as the liver is one of the major targets in AAV-mediated gene therapy (Manno, C. S. et. al. 2006 Nat. Med. 12:342-347). Purified peritoneal macrophages and hepatic Kupffer cells were stimulated with AAV2-lacZ or Ad-lacZ and assayed for the secretion of IFN-γ and IL-6. Our data indicated that freshly isolated Kupffer cells and macrophages could not produce significant levels of IFN-γ or IL-6 upon AAV infection in contrast to the infection with adenovirus (FIG. 2). These results further confirm that AAV mainly activates pDC, but not non-pDCs, to produce type I IFNs.

### Example 3

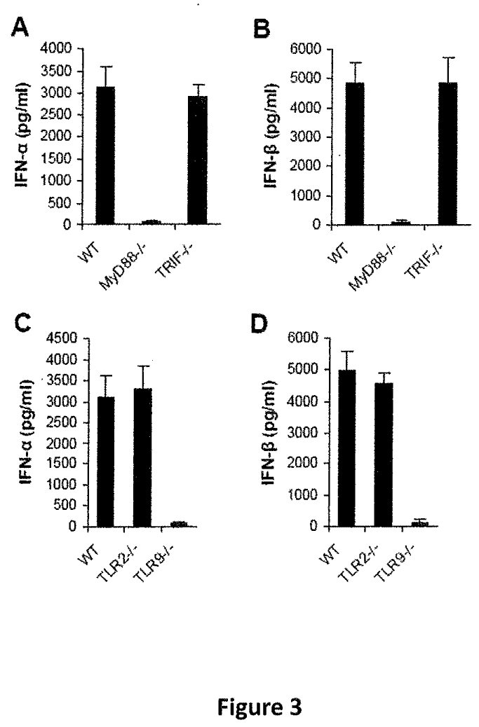

Innate immune recognition of AAV2 is mediated by TLR9 and dependent on MyD88. We next investigated whether TLRs were involved in the induction of type I IFNs by pDCs upon AAV infection. Since all TLR signaling is mediated by MyD88 and/or TRW (Akira, S. et al. 2006 Cell 124:783-801), pDCs deficient for MyD88 (MyD88−/−) or TRIF (TRIF−/−) were tested for their ability to produce type I IFNs upon AAV infection. pDCs generated from bone marrow cells of MyD88−/− or TRIF−/− C57BL/6 mice were stimulated with AAV2-lacZ and assayed for type I IFN secretion. The production of both IFN-α (FIG. 3A) and IFN-β (FIG. 3B) by MyD88−/− pDCs was abolished, whereas that by TRIF−/− pDCs was not affected compared to the wild type (WT) pDCs (FIG. 3). These data indicate that the production of type I IFNs by pDCs in response to AAV2 was TLR-mediated and dependent on MyD88.

### Example 4

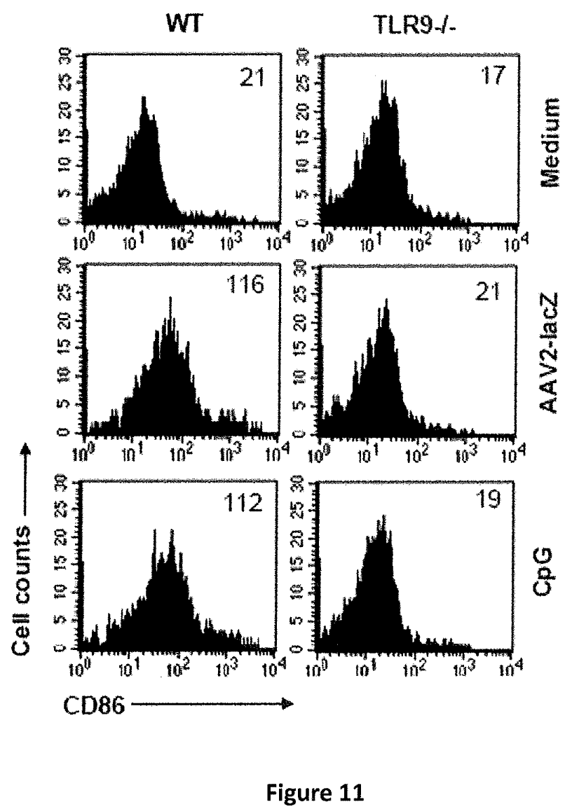

pDC maturation upon AAV infection is mediated by the TLR9/MyD88 signaling pathway. Which TLR then mediated the MyD88-dependent production of type I IFNs by pDCs upon AAV infection? Among all TLRs characterized to date; only TLR7, TLR8 and TLR9 are known to mediate MyD88-dependent production of type I IFNs (Akira, S. et al. 2006 Cell 124:783-801). Since the known ligands for TLR7 and TLR8 are single-stranded RNA, and AAV is a single-stranded DNA virus, we hypothesized that a likely candidate to mediate induction of type I IFNs by pDCs was TLR9. To test this, we examined whether pDCs generated from TLR9−/− C57BL/6 mice secreted type I IFNs upon AAV infection. Similar to MyD88−/− pDCs (FIG. 3A, B), TLR9−/− pDCs stimulated with AAV2-lacZ failed to secrete IFN-α (FIG. 3C) or IFN-β (FIG. 3D), whereas production of these type I IFNs was not affected in TLR2−/− or WT pDCs upon AAV infection. We further showed that the up-regulation of CD86 was abolished in TLR9−/− pDCs compared to the WT control (FIG. 11), suggesting pDC maturation upon AAV2 infection is also dependent on TLR9 signaling. Taken together, these observations indicate that pDC maturation and production of type I IFNs upon AAV infection is mediated by the TLR9-MyD88 pathway. Similar results were obtained with pDCs generated from WT, MyD88−/− or TLR9−/− BALB/c mice (data not shown).

### Example 5

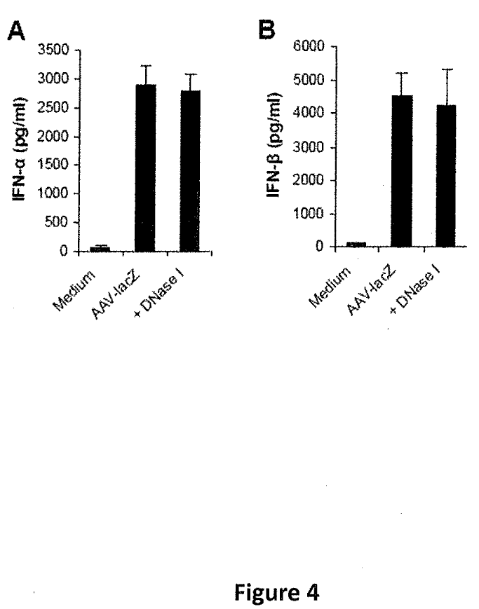

Viral DNA is responsible for TLR9 stimulation in pDCs. The TLR9-dependent sensing of AAV also suggests that the ligand for TLR9 recognition is viral DNA. Since the AAV stock was produced by transfecting 293 cells with the vector and the helper plasmids, followed by purification with heparin affinity chromatography as described (Auricchio, A. et al. 2001 Hum. Gene Ther. 12:71-76), there was a concern about potential contamination of the purified AAV with residual plasmid DNA, which may account for the observed TLR9-dependent activation of pDCs. To rule out this possibility, AAV2-lacZ was treated again with DNase I and used for stimulating pDCs. Our data showed that pDCs stimulated with DNase I-treated AAV produced similar levels of IFN-α (FIG. 4A) and IFN-β (FIG. 4B) to those with untreated AAV, suggesting that viral DNA, but not the contaminating plasmid DNA, is responsible for the stimulation of TLR9 in pDCs.

### Example 6

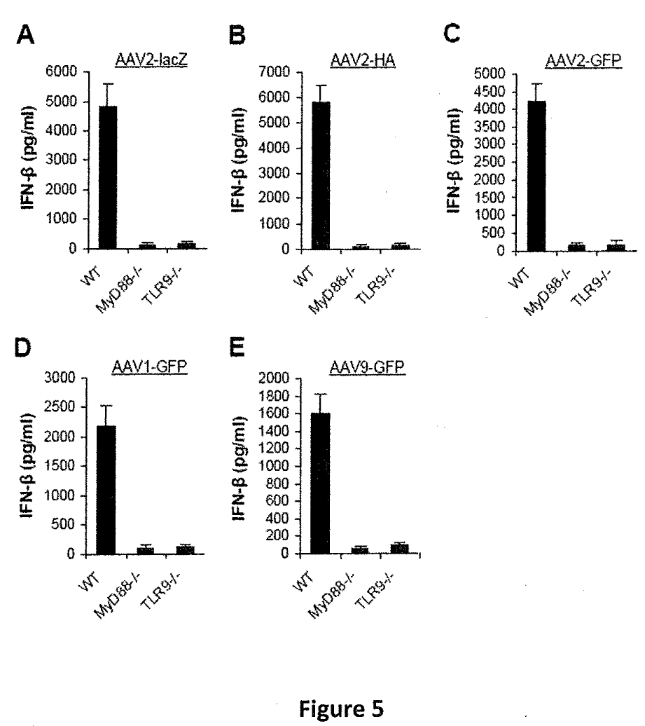

Activation of the TLR9-MyD88 pathway by AAV is independent of the nature of the transgene or AAV serotypes. We next sought to determine whether the innate immune recognition of AAV2 encoding other transgenes was also dependent on the TLR9-MyD88 pathway. To address this question, WT, MyD88−/−, or TLR9−/− pDCs were stimulated with AAV2-lacZ, AAV2 encoding influenza hemagglutinin (AAV2-HA), or GFP (AAV2-GFP) and examined for the secretion of IFN-β. Similar to AAV2-lacZ (FIG. 5A), neither AAV2-HA (FIG. 5B), nor AAV2-GFP (FIG. 5C) could stimulate TLR9−/− or MyD88−/− pDCs to produce IFN-β, suggesting that the activation of the TLR9-MyD88 pathway by AAV is independent of the nature of the transgene that AAV encodes.

### Example 7

Innate immune activation by other AAV serotypes is also dependent on the TLR9-MyD88 signaling pathway. Although AAV2 is the most extensively studied AAV vectors, other serotypes of AAV have also been developed as vectors for gene therapy (Wu, Z. et al. 2006 Mol. Ther. 14:316-327). We thus examined whether the innate immune recognition of other AAV serotypes was also mediated by the TLR9-MyD88 pathway. WT, MyD88−/−, or TLR9−/− pDCs were infected with AAV2-GFP, AAV1-GFP, or AAV9-GFP and examined for the secretion of IFN-β. Although WT pDCs infected with AAV1-GFP (FIG. 5D) or AAV9-GFP (FIG. 5E) produced lower levels of IFN-β than those infected with AAV2-GFP (FIG. 5C), our data showed that similar to AAV2-GFP (FIG. 5C), no type I IFN secretion was detected in TLR9−/− or MyD88−/− pDCs upon stimulation with AAV1-GFP (FIG. 5D), or AAV9-GFP (FIG. 5E). These results suggest that innate immune activation by other AAV serotypes is also dependent on the TLR9-MyD88 pathway:

### Example 8

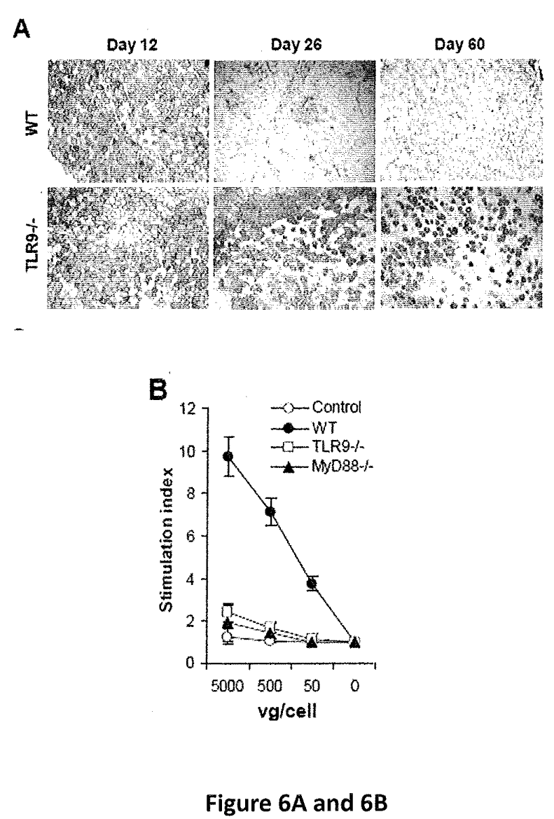

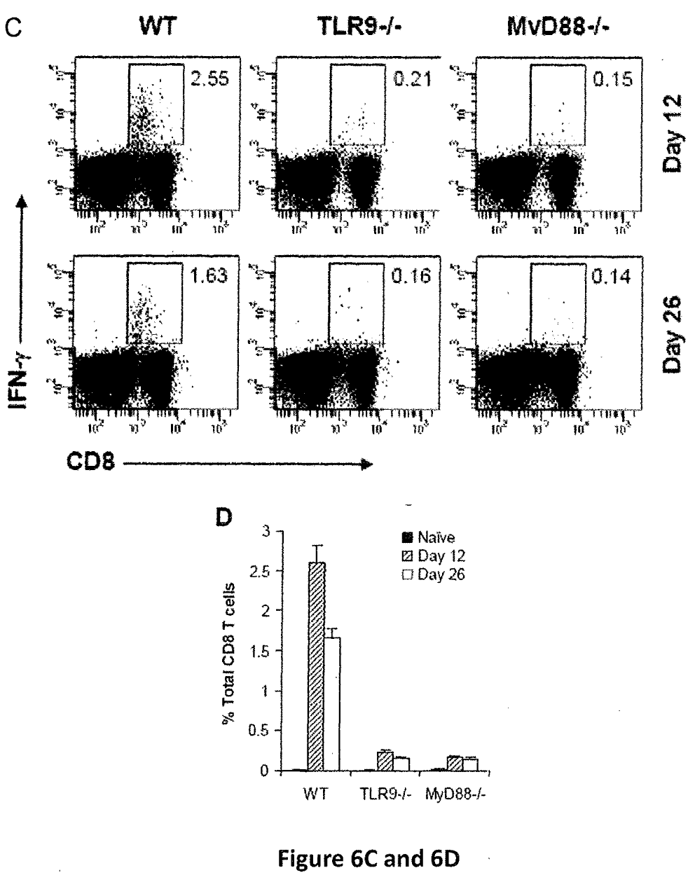

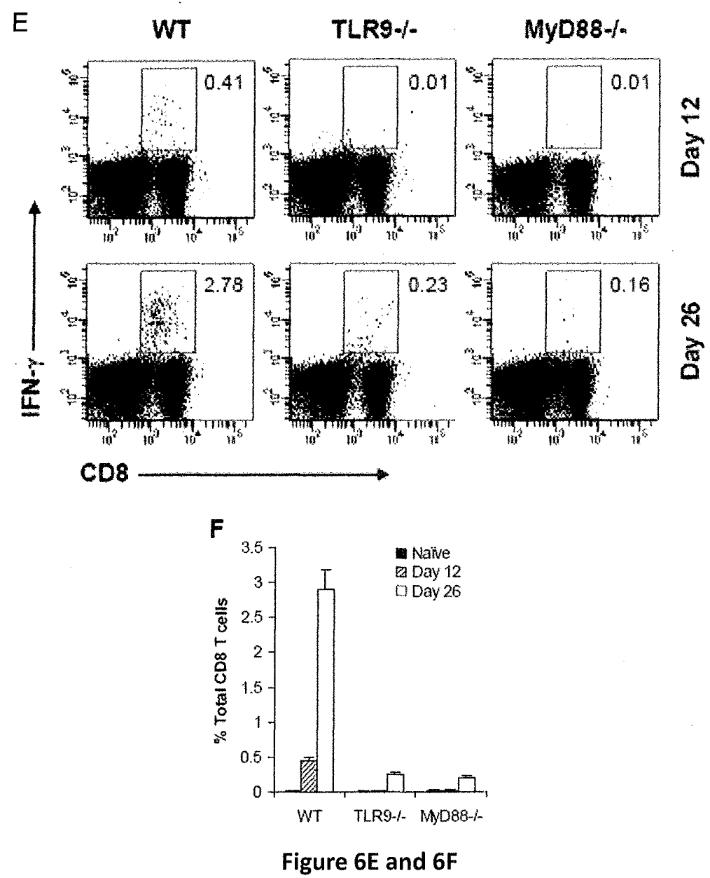

The TLR9-MyD88 pathway is critical for CD8 T cell responses to the transgene product and the AAV capsid, and the loss of transgene expression in vivo. We next determined the biological significance of the TLR9-MyD88 pathway in adaptive immune responses to AAV vectors in vivo. To address this question, we utilized a murine model of skeletal muscle-mediated gene transfer because the skeletal muscle is widely considered as a target for AAV-mediated gene therapy in vivo. AAV2-HA was injected intramuscularly into WT, TLR9−/−, or MyD88−/− BALB/c mice. 12 days later, high levels of HA expression were detected in the skeletal muscles of all mice (FIG. 6A). However, the transgene expression was transient as HA expression was significantly reduced by day 26 after injection and completely cleared by day 60 (FIG. 6A). This was consistent with the previous observation that AAV-mediated HA expression in skeletal muscles of BALB/c mice is transient (Sarukhan, A. et al. 2001 J. Virol. 75:269-277). In contrast, the HA expression was stable in TLR9−/− (FIG. 6A) or MyD88−/− (data not shown) mice. At day 26, splenocytes were analyzed for virus-specific T cell activation using the standard T cell proliferation assay by ³H-thymidine incorporation upon in vitro re-stimulation with different doses of AAV2-HA. In WT mice, AAV infection resulted in robust T cell activation, whereas in TLR9−/− or MyD88−/− mice, T cell activation was significantly diminished (p<0.001, FIG. 6B). These results suggest that the TLR9-MyD88 pathway is critical for the activation of AAV-specific T cells, leading to the loss of transgene expression in vivo.

### Example 9

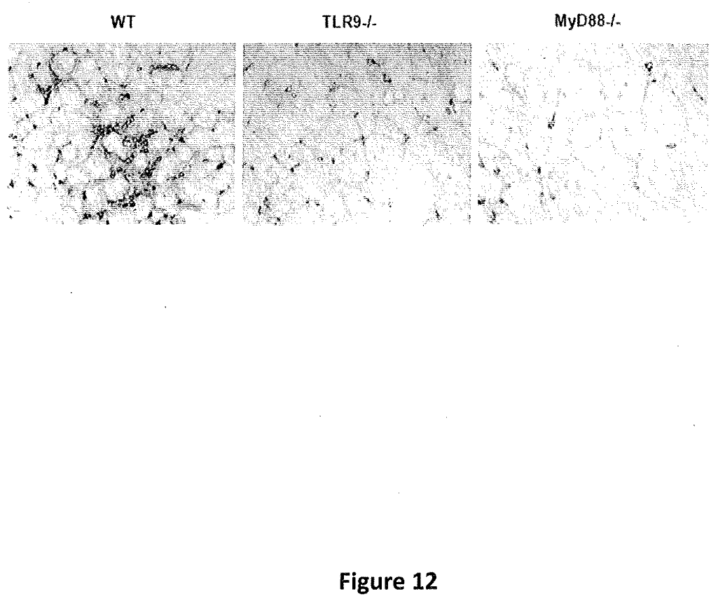

An intact TLR9-MyD88 signaling pathway is required for the activation of both AAV capsid- and transgene expression in vivo. Since the proliferation assay measures total T cell responses to AAV, we further examined cytotoxic CD8 T cell responses to the HA transgene and the AAV2 capsid using IFN-γ intracellular staining assay upon in vitro re-stimulation with immunodominant epitope peptides specific for HA and the AAV capsid, respectively. In WT mice, AAV infection resulted in activation of both capsid- and HA-specific CD8 T cell responses, however, with a different kinetics: robust capsid-specific CD8 T cell response was observed at both days 12 and 26 (FIG. 6C, D), whereas high levels of HA-specific CD8 T cells were detectable only at day 26 (FIG. 6E, F). This difference in kinetics may reflect availability of different antigens for presentation by DCs: AAV-mediated transgene expression in vivo usually takes 1-2 weeks, whereas the input AAV capsid is readily available upon viral infection. Despite this difference, in TLR9−/− or MyD88−/− mice, CD8 T cell responses to both the AAV capsid (FIG. 6C, D) and HA (FIG. 6E, F) were significantly (p<0.001) diminished compared to the WT control. This was associated with a reduction in CD8 T cell infiltration into the infected muscles of TLR9−/− or MyD88−/− mice compared to the WT group (FIG. 12). These results indicate that an intact TLR9-MyD88 pathway is required for the activation of both AAV capsid- and transgene product-specific CD8 T cells, and the loss of transgene expression in vivo.

### Example 10

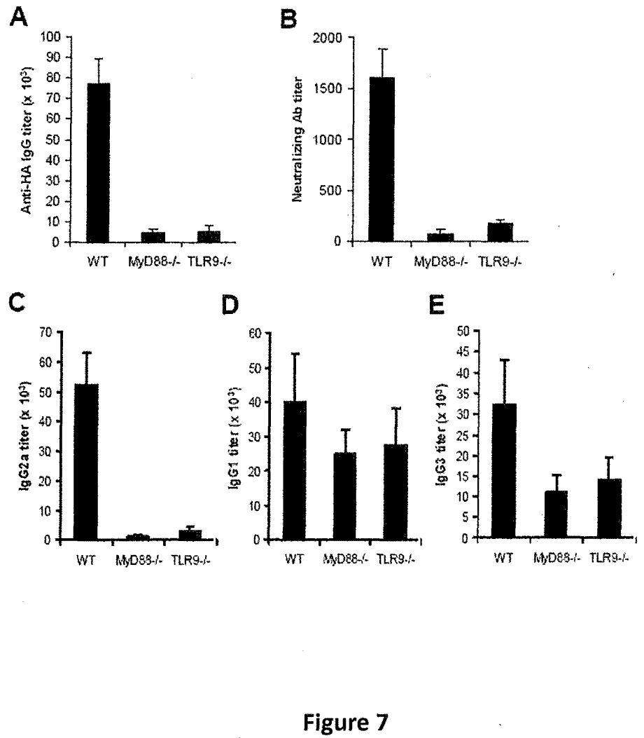

The transgene-specific and AAV-neutralizing antibody responses are also dependent on the TLR9-MyD88 pathway. We next investigated if the TLR9-MyD88 pathway also influenced adaptive B cell response to AAV infection. WT, TLR9−/− or MyD88−/− mice were injected intramuscularly with 1×10¹¹ vg of AAV2-HA, and serum samples were harvested 36 days later and assayed for the presence of anti-HA antibody and the neutralizing antibody to AAV. Sera from WT mice infected with AAV2-HA were found to contain high titers of anti-HA antibody (FIG. 7A) and neutralizing antibody to AAV (FIG. 7B). However, both the anti-HA antibody response (FIG. 7A) and neutralizing antibody titers (FIG. 7B) were significantly (p<0.001) diminished in AAV2-HA-infected TLR9−/− and MyD88−/− mice compared to the WT control. We further analyzed AAV vector-specific Ig isotypes by ELISA. The results revealed that a similar degree of reduction in AAV-specific IgG2a (indicative of Th1-dependent B cell response) titers in TLR9−/− and MyD88−/− mice, compared to the WT control (FIG. 7C). However, only moderate reduction of AAV-specific IgG1 (indicative of Th2-dependent B cell response, FIG. 7D) and IgG3 (indicative of Th-independent B cell response, FIG. 7E) titers was noted in TLR9−/− and MyD88−/− mice. Thus, the formation of transgene-specific and AAV-neutralizing antibodies is also dependent on an intact TLR9-MyD88 signaling pathway.

### Example 11

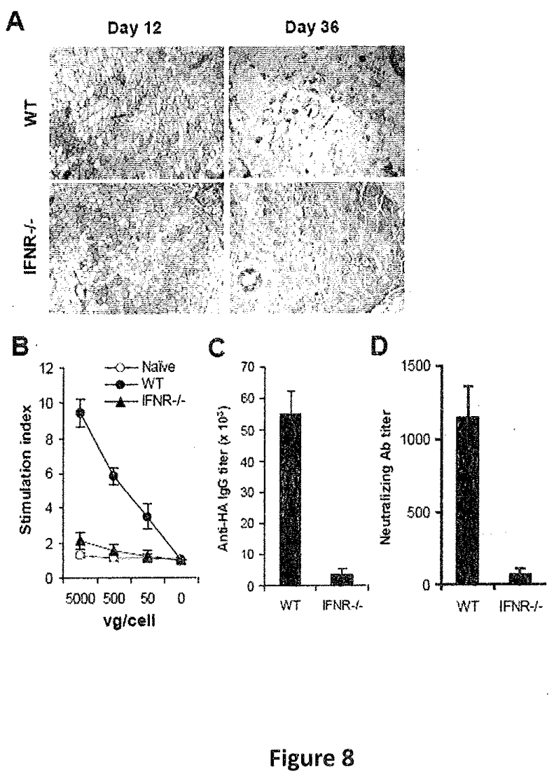

Type I IFNs are required for adaptive immune responses to AAV. How does the TLR9-MyD88 innate immune pathway regulate adaptive immune responses to AAV? The TLR9-dependent production of mainly type I IFNs by pDCs upon AAV infection suggested that type I IFNs may play a key role in promoting adaptive immune responses to AAV in vivo. To test this hypothesis, we examined if adaptive immune responses to AAV was dependent on type I IFNs. AAV2-HA (1×10¹¹ vg) was injected intramuscularly into WT mice or mice deficient for the IFN-α and IFN-β receptor (IFNR−/−) and examined for HA expression 12 and 36 days after injection. High levels of HA expression were detected in the skeletal muscles of WT and IFNR−/− mice 12 days after infection (FIG. 8A). By day 36, significant loss of HA expressing muscle fibers was detected in WT mice. By contrast, the expression of HA remained stable in IFNR−/− mice (FIG. 8A). This corresponded to a significant (p<0.001) reduction in AAV-specific T cell activation in IFNR−/− mice compared to the WT control (FIG. 8B). In addition, both anti-HA antibody (FIG. 8C) and AAV-neutralizing antibody (FIG. 8D) titers were significantly (p<0.001) diminished in IFNR−/− mice. Taken together, these data indicated that type I IFNs played a pivotal role in the TLR9-MyD88 dependent adaptive immune responses to AAV vectors in vivo.

### Example 12

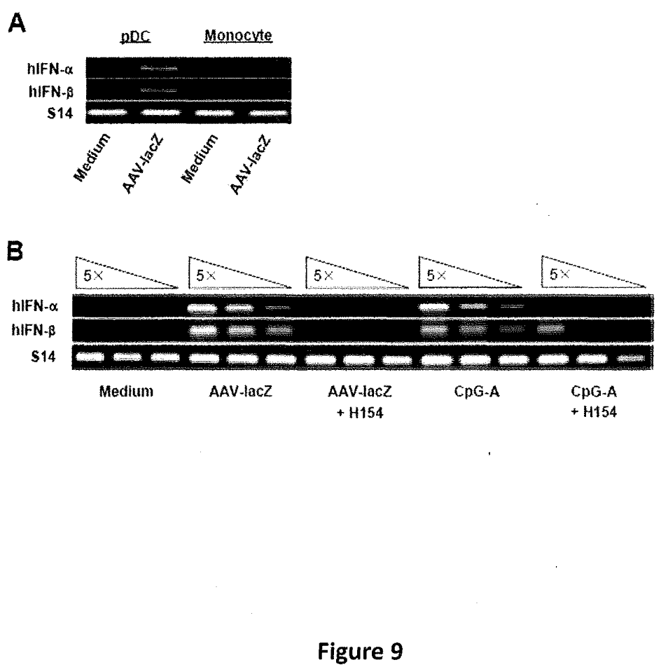

AAV also activates human pDCs in a TLR9-dependent manner. The critical role for the TLR9-MyD88 pathway in innate and adaptive immune responses to AAV vectors suggested that strategies to interfere with the TLR9-MyD88 pathway may improve the outcome of AAV-mediated gene therapy in humans. As a first step to testing this strategy, here we examined if AAV also activated human pDCs via TLR9. Human pDCs and monocytes were purified from peripheral blood mononuclear cells (PBMCs) and stimulated with AAV2-lacZ for 18 h. Cells were then harvested and total RNA was examined for the expression of human IFN-α (hIFN-α) and hIFN-β by RT-PCR. Consistent with the observation in mice, AAV-stimulated human pDCs, but not non-pDCs such as monocytes, induced the expression of hIFN-α and hIFN-β (FIG. 9A). To test whether activation of human pDCs by AAV was also mediated by TLR9, purified pDCs were pre-treated with a TLR9 antagonist, H154 oligodeoxynucleotide (ODN) (Yamada, H. et al. 2002 J. Immunol. 169:5590-5594), followed by the stimulation with AAV2-lacZ, and cellular RNA was analyzed for the induction of hIFN-α and hIFN-β by semi-quantitative RT-PCR. Our data showed that pretreatment with H154 ODN completely blocked the induction of hIFN-α and hIFN-β upon AAV infection, compared to the untreated controls (FIG. 9B). Similarly, pretreatment with H154 ODN also abrogated the induction of hIFN-α and hIFN-β by a TLR9 agonist, CpG-A ODN (Peng, G. et al. 2005 Science 309:1380-1384), confirming the specificity of TLR9 blocking by H154 ODN (FIG. 9B). Thus, these results indicate that the activation of human pDCs by AAV is also mediated by TLR9 and suggest a strategy for TLR9 blockade to blunt AAV-induced innate immune response.

Discussion

The adaptive immune responses to AAV represent a significant hurdle in clinical application of AAV vectors for gene therapy. Recent developments have suggested a critical role for the innate immunity in promoting adaptive immune responses. A major unanswered question is how AAV activates the innate immune pathway. In this study, we demonstrated that AAV activated pDCs, but not non-DCs such as cDCs and macrophages, to produce type I IFNs through the TLR9-MyD88 pathway. In vivo, the TLR9-MyD88 innate immune pathway was required for the activation of CD8 T cell responses to both the transgene product and the AAV capsid, leading to the loss of transgene expression. Furthermore, the formation of antibodies to the transgene product and the AAV vector was also critically dependent on this pathway. In addition, we showed that TLR9-dependent activation of adaptive immune responses to AAV was mediated by type I IFNs, and that AAV also activated human pDCs to induce type I IFNs via TLR9.

Our finding that similar to adenoviral vectors, AAV predominantly activated pDCs via the TLR9-MyD88 pathway to secrete type I IFNs is in line with previous observations that pDCs are the most potent type I IFN producers and secrete mainly type I IFNs upon TLR9 stimulation (Zhu, J. et al. 2007 J. Virol. 81:3170-3180; Colonna, M. et al. 2004 Nat. Immunol. 5:1219-1226). The mechanism(s) underlying this pDC-specific involvement of the TLR9-MyD88-type I IFN pathway remains incompletely defined. Studies have shown that stimulation of TLR9 with CpG DNA in pDCs activates MyD88, which then interacts with IRAK1 and TRAF6, leading to the activation of IKKα and IRF7 and the production of type I IFNs (Hoshino, K. et al. 2006 Nature 440:949-953; Uematsu, S. et al. 2007 J. Biol. Chem. 282:15319-15323). However, this pathway is not operative in non-pDCs such as cDCs. Furthermore, pDCs express high levels of IRF7 and osteopontin, both of which are critical for the induction of type I IFNs (Izaguirre, A. et al. 2003 J. Leukoc Biol. 74:1125-1138; Shinohara, M. L. et al. 2006 Nat. Immunol. 7:498-506). In addition, CpG DNAs are retained longer in pDC endosomes where TLR9 resides, but are rapidly transferred to lysosomes for degradation in non-pDCs (Honda, K. et al. 2005 Nature 434:1035-1040; Guiducci, C. et al. 2006 J. Exp. Med. 203:1999-2008). Indeed, we have found that pDCs are poorly transduced by adenoviral vectors (Zhu, J. et al. 2007 J. Virol. 81:3170-3180), which may be related to the preferential retention of CpG-containing viral DNA in the endosome.

The TLR9-dependent recognition of AAV also suggests that the ligand for TLR9 recognition is viral DNA. How is the encapsidated single-stranded AAV DNA exposed in endosomes for its recognition by TLR9? It has been shown that following clathrin-dependent or independent internalization, transducing AAV is routed through the endosomal compartment, where pH dependent penetration of endosomes by the virus occurs (Dollar, A. M. et al. 2001 J. Virol. 75:1824-1833). Studies with other viruses have suggested that the highly acidified endosomal compartment which contains abundant proteolytic degradation enzymes, may damage viral particles and release some viral DNA for recognition by TLR9 (Kawai, T. et al. 2006 Nat. Immunol. 7:131-137; Crozat, K. et al. 2004 Proc. Natl. Acad. Sci USA 101:6835-6836). This process is independent of viral transduction and viruses that do not normally replicate in pDCs, as well as defective viral particles or inactivated virus, can also be detected. Even viruses neutralized by antibody or complement can be taken up via Fc or complement receptors and subject to TLR recognition within endosomes (Wang, J. P. et al. 2007 J. Immunol. 178:3363-3367). Similar to AAV2, other serotypes of AAV including AAV1 and AAV9 also activate pDCs via the TLR9-MyD88 pathway. However, pDCs infected with AAV1 or AAV9 appear to induce lower levels of type I IFNs than those with AAV2, suggesting different serotypes of AAV may differ in activating the innate immune system. It remains to be defined whether this reflects a difference in endosome targeting and/or processing of AAV. Thus, further investigation is needed to define endosomal sensing of AAV by TLR9.

Whether capsid components of AAV can activate innate immune responses is unknown. A recent report has suggested that complement components might interact with AAV capsid to enhance the stimulation on macrophage by AAV in vitro (Zaiss, A. K. et al. 2008 J. Virol. 82:2727-2740). However, cytokine secretion upon AAV infection is not compromised in mice deficient for complement components in vivo, suggesting that such an interaction may not exist in vivo (Zaiss, A. K. et al. 2008 J. Virol. 82:2727-2740). Thus, the role of complement components in innate immune response to AAV in vivo remains uncertain.

The observation that very low levels of type I IFNs and pro-inflammatory cytokines were produced by non-pDCs upon AAV infection is in striking contrast to adenoviral vectors. Since production of pro-inflammatory cytokines and type I IFNs by pDCs stimulated with adenoviral vectors is mediated by a TLR-independent pathway through cytosolic sensing of double-stranded adenoviral DNA (Zhu, J. et al. 2007 J. Virol. 81:3170-3180; Nociari, M. et al. 2007 J. Virol. 81:4145-4157), these data suggest that the single-stranded AAV genome may not activate this pathway efficiently in non-pDCs. Indeed, it is believed that the ligand for the yet-to-be-identified cytosolic DNA sensor is double stranded B-form DNA derived from many microbes (Stetson, D. B. et al. 2006 Immunity 24:93-103; Ishii, K. J. et al. 2006 Nat. Immunol. 7:40-48). The very low levels of cytokines produced by non-pDCs upon AAV infection may explain a lack of strong inflammatory responses documented in numerous models of AAV-mediated gene therapy in vivo (Zaiss, A. K. et al. 2005 Curr. Gene Ther. 5:323-331). As pDCs mainly reside in the spleen and other secondary lymphoid organs (Colonna, M. et al. 2004 Nat. Immunol. 5:1219-1226), the lack of strong type I IFN or pro-inflammatory cytokine responses from non-pDCs upon AAV infection may also explain a recent observation that robust transcriptome responses, including the induction of a cluster of type I IFN-related genes, associated with adenoviral vectors by microarray analysis of the liver RNA were not observed with AAV vectors (McCaffrey, A. P. et al. 2008 Mol. Ther. 16:931-941). Taken together, the above observations are consistent with the notion that AAV is a weak immunogen compared to adenoviral vectors.

The biological significance of the TLR9-MyD88-type I IFN pathway in innate sensing of AAV by pDCs lies in its critical role in the activation of adaptive T and B cell responses to the transgene product and the AAV vector. Our results indicate that an intact TLR9-MyD88 pathway is required for the activation of both AAV capsid- and transgene product-specific CD8 T cells. The lack of CD8 T cell responses early after infection (day 12) in TLR9−/− or MyD88−/− mice suggests that the TLR9-MyD88 pathway is critical for CD8 T cell priming. The observation that the kinetics of transgene-specific CD8 T cell response is closely associated with the loss of transgene expression (FIG. 6A, E, F), suggests that transgene-specific CD8 T cells may be critical for the elimination of the transduced cells in vivo. This is very similar to a recent observation that lentiviral vectors activate pDCs via TLR7 to secrete type IFNs, which is required for subsequent activation of CTLs against the transgene product (Brown, B. D. et al. 2007 Blood 109:2797-2805). However, since the TLR9 pathway also regulates the activation of capsid-specific CD8 T cells, we cannot rule out the role of capsid-specific T cells in eliminating AAV-transduced cells in viva.

The mechanism(s) underlying type I IFN-dependent adaptive immune responses to AAV requires further investigation. Studies in other models have shown that type I IFNs can promote DC maturation and function (Honda, K. et al. 2003 Proc. Natl. Acad. Sci. USA 100:10872-10877; Hoebe, K, et al. 2003 Natl. Immunol. 4:1223-1229). Type I IFNs have also been shown to enhance cross-presentation by DCs (Le Bon, A. et al. 2003 Nat. Immunol. 4:1009-1015). This observation is particularly relevant to AAV infection since the activation of both transgene- and viral capsid-specific CTL responses are thought to be dependent on cross-presentation by MHC class I (Manning, W. C. et al. 1997 J. Virol. 71:7960-7962; Sarukhan, A. et al. 2001 J. Virol. 75:269-277; Vandenberghe, L. H. et al. 2006 Nat. Med. 12:967-971; Wang, Z. et al. 2007 Hum. Gene Ther. 18:18-26; Wang, L. et al. 2007 Hum. Gene Ther. 18:185-194; Li, C. et al. 2007 J. Virol. 81:7540-7547). Furthermore, we have recently shown that direct type I IFN signaling is required for the survival of activated T cells in response to vaccinia viral infection (Quigley, M, et al. 2008 J. Immunol. 180:2158-2164). Type I IFNs are also critical for multiple stages of adaptive B cell response to adenovirus, and the generation of protective neutralizing antibodies to adenovirus critically depends on type I IFN signaling on both CD4 T cells and B cells (Zhu, J. et al. 2007 J. Immunol. 178:3505-3510). Thus, future studies should focus on defining mechanisms by which type I IFNs promote adaptive immune responses to AAV.

Our observation that human pDCs can also be activated by AAV to induce type I IFNs via TLR9, suggests that the TLR9 pathway might also be important in regulating adaptive immune responses to AAV in humans. However, studies have shown that the induction of adaptive immune responses to AAV is influenced by many factors including host species and the pre-existing immunity to AAV (Vandenberghe, L. H. et al. 2007 Curr. Gene Ther. 7:325-333). Thus, additional studies in non-human primates as well as in human clinical trials are required to define the role of TLR9 innate immune pathway in the activation of adaptive immune responses to AAV in humans.

The route of administration has also played a role in adaptive immune responses to AAV. Studies in murine models have shown that hepatic delivery of AAV often results in immune tolerance to the transgene product (Ge, Y. et al. 2001 Blood 97:3733-3737; Xiao, O. et al. 2000 Mol. Ther. 1:323-329). It is not clear whether this is a result of defective innate immune activation in the liver (e.g., insufficient pDCs or lack of interaction of AAV with pDCs), or the existence of immunosuppressive cell types such as regulatory T cells and Kupffer cells, and/or immunosuppressive cytokines such as IL-10 in the hepatic microenvironment (Erhardt, A. et al. 2007 Hepatology 45:475-485; You, Q. et al. 2008 Hepatology 48:978-990). Thus, it will be important to delineate factors that influence immune responses to AAV in the liver.

In conclusion, our study reveals that AAV activates the innate immunity through the TLR9-MyD88 pathway in pDCs, which leads to the production of type I IFNs. In vivo, this innate immune pathway plays a critical role in the activation of CD8 T cell responses to both the transgene product and the AAV capsid, and the formation of anti-transgene and AAV-neutralizing antibodies. Furthermore, AAV also activates human pDCs to produce type I IFNs in a TLR9-dependent fashion. These results suggest that strategies targeted to interfere with the TLR9-MyD88-type I IFN signaling pathway may improve the safety and efficacy of AAV vectors for gene therapy in humans.

Materials and Methods

Mice. C57BL/6 and BALB/c mice were purchased from the Jackson Laboratory. TLR2−/−, TLR9−/−, MyD88−/−, and TRIF−/− mice on C57BL/6 background were kindly provided by Shizuo Akira (Osaka University, Osaka, Japan). IFN-αβR−/− mice (Muller U. et al. 1994 Science 264:1918-1921) on 129/Sv background and their normal control 129/Sv mice were obtained from B & K Universal. TLR9−/− and MyD88−/− mice have been backcrossed onto BALB/c background for more than nine generations in our animal facility. Groups of 7-10 wk-old mice were selected for this study. All experiments involving the use of mice were done in accordance with protocols approved by the Animal Care and Use Committee of Duke University.

Recombinant AAV. Recombinant AAV2 encoding influenza hemagglutinin (AAV2-HA), lac Z (AAV2-lacZ) or GFP (AAV2-GFP) under the control of CMV promoter were generated with a helper virus-free system (Stratagene) by transfecting 293 cells (which stably express the adenovirus E1 gene) with three plasmids as described (Matsushita, T. et al. 1998 Gene Ther. 5:938-945; Xiao, X. et al. 1998 J. Prof. 72:2224-2232). These three plasmids are pHelper, encoding E2A, E4 and VA RNA genes of adenovirus; pAAV-RC, encoding AAV2 rep and cap genes; and vector plasmid pAAV-CMV-HA, lacZ or GFP. Purification of AAV was done by heparin affinity chromatography as described (Auricchio, A, et al 2001 Hum. Gene Ther. 12:71-76). Briefly, cells were disrupted by freezing and thawing two times and cell lysates were incubated with 40 μg/ml of DNase I and RNase A (Roche Biochemicals) for 30 min at 37° C. After centrifugation, the supernatants were then incubated with 0.5% deoxycholic acid (Sigma) for 30 min at 37° C., followed by filtration through a 5-μm and a 0.8-μm pore size filter (Millipore) sequentially. The cleared supernatants were then loaded onto a heparin column. After washing twice with PBS, pH7.4, plus 0.1 M NaCl, the virus was eluted with PBS, pH7.4, plus 0.4 M NaCl. The eluate was concentrated with a Millipore Biomax-100K NMWL filter device by centrifugation. The viral genome (vg) titer was determined by a CMV promoter-specific quantitative real time PCR procedure.

For the production of recombinant AAV1-GFP and AAV9-GFP, pAAV-CMV-GFP and pHelper were co-transfected into 293 cells along with a chimeric packaging plasmid in which the AAV2 rep gene was fused to AAV1 and AAV9 capsid genes, respectively. Cells were harvested, sonicated and treated with DNase I and RNase A. The resultant AAV1-GFP and AAV9-GFP viral particles were purified twice by CsCl density gradient ultracentrifugation as described (Auricchio, A. et al. 2002 J. Cling. Invest. 110:499-504).

Murine DC culture. pDCs were generated as described (Zhu et al. 2007 J. Virol. 81:3170-3180). Briefly, bone marrow cells were harvested from femurs and tibiae of mice and cultured in the presence of 200 ng/ml of Flt-3 ligand (R & D Systems) for 9 days. For generation of cDCs, bone marrow cells were in the presence of mouse GM-CSF (1,000 U/ml) and IL-4 (500 U/ml) (R & D Systems) for 5 days as described (Yang, Y. et al. 2004 Nat. Immunol. 5:508-515). pDCs and cDCs were stained with anti-B220-FITC and anti-CD11c-PE and purified by FACS sorting. Purified cells were then stimulated with various agents at a density of 1×10⁶ cells/ml.

Isolation of murine splenic DCs, macrophages and Kupffer cells. Splenic DC isolation was performed as described (Zhu et al. 2007 J. Virol. 81:3170-3180). After perfusion with Liberase CI (Roche Biochemicals), single cell suspensions were subjected to 30% BSA gradient, and the interface DC fraction was collected and stained with anti-B220-FITC and anti-CD11c-biotin followed by streptavidin-microbeads (Miltenyi Biotec). CD11c+DCs were purified by positive selection by microbeads and subjected to FACS sorting into pDCs (CD11c+B220+) and cDCs (CD11c+B220−). Macrophages were isolated from the peritoneal cavity of mice 3 days after intraperitoneal injection of 2.5 ml of 3% thioglycollate as described (Lund, J. M. et al. 2004 Proc. Natl. Acad. Sci. 101:5598-560). Kupffer cells were isolated from mouse livers as described (Zhu et al. 2007 J. Virol. 81:3170-3180). After perfusion in situ via portal vein with collagenase, single cell suspensions were subjected to gradient centrifugation with 11.5% OptiPrep solution. Kupffer cell fraction was collected from the interface and purified by FACS sorting for F4/80 positive cells. Purified splenic CD11c+ DCs, macrophages and Kupffer cells were stimulated with various agents at a density of 2.5×10⁵ cells/ml.

Detection of cytokines by ELISA. Production of IL-6, TNF-α, IFN-α and IFN-β by DCs in response to various stimuli was detected in culture supernatants by ELISA kits according to manufacturer's standard protocols. IL-6 and TNF-α ELISA kits were purchased from Endogen Pierce. IFN-α and IFN-β kits were obtained from PBL Biomedical Laboratories.

In vivo delivery of recombinant AAV. 1×10¹¹ vg of AAV-HA in 25 μl was injected into tibialis anterior muscles of mice. Mice were sacrificed at indicated time points for histological and immunological assays. All animals that received recombinant virus survived to the time of necropsy.

Proliferation assay. T cells were isolated from splenocytes using CD5-microbeads (Miltenyi Biotec). CD5+ T cells (2×10⁵) were co-cultured with irradiated (3000 rad) naïve splenocytes (2×10⁵) in the presence of AAV2-HA at 0, 50, 500, or 5000 vg/cell for 72 hr in triplicates. Cultures were pulsed with 1 μCi per well of ³H-thymidine. 16-20 hr after pulsing, plates were harvested using a 96-well cell harvester and the 3H-thymidine incorporation was counted using a 1205 Betaplate scintillation counter (Wallace).

Antibodies and flow cytometry. All antibodies used for FACS were purchased from BD Biosciences. FACSCanto (BD Biosciences) was used for flow cytometry event collection and data were analyzed using FACS DIVA and CELLQuest software (BD Biosciences).

For intracellular IFN-γ staining, splenocytes were stimulated with 2 μg/ml of Ld-restricted AAV-2 capsid epitope peptide (372VPQYGYLTL380) (Sabatino, D. E. et al. 2005 Mol. Ther. 12:1023-1033) or Kd-restricted HA epitope peptide (518IYSTVASSL526) (Yang, Y. et al. 2004 Nat. Immunol. 5:508-515), and 5 μg/ml of GolgiPlug (BD Biosciences) for 5 hr. After washing, cells were stained with anti-CD8 (Clone 53-6.7) and permeabilized to detect IFN-γ intracellularly with anti-IFN-γ (Clone XMG1.2) using the Cytofix/Cytoperm kit (BD Biosciences) as previously described (Yang, Y. et al. 2004 Nat. Immunol. 5:508-515).

Immunohistochemical staining. Frozen sections (5 μm) of muscles was fixed with acetone, air dried and rehydrated in PBS. After blocking with 20% goat-serum in PBS, sections were stained with biotinylated anti-HA or anti-CD8 mAb, followed by ABC kit (Vector Laboratories) as described (Huang, X. et al. 2004 Eur. J. Immunol. 34:1351-1360).

Neutralizing antibody assay. Neutralizing antibody titers were analyzed by assessing the ability of serum antibody to inhibit transduction of AAV2-LacZ into AAV permissive HT1080 cell line. 60-70% confluent HT1080 cells in 96-well plates (2×10⁴ cells per well) were treated with 0.2 ml of 240 mM of hydroxyurea and 3 mM of sodium butyrate for 6 hr at 37° C. Serum samples were incubated at 56° C. for 30 min and then diluted in 2-fold steps starting from 1:20 or 1:50. Serial dilutions of sera were pre-incubated with 2.5×10⁸ vg of AAV2-lacZ in a 100 μl total volume for 1 hr at 37° C., and added to pre-treated cell cultures. Cells were fixed and analyzed for lacZ expression by X-gal staining on the following day as described (Zhu, J. et al. 2007 J. Immunol. 178:3505-3510). All of the cells stained blue in the absence of serum samples. The titer of neutralizing antibody for each sample was reported as the highest dilution with which less than 50% of cells stained blue.

AAV2-specific antibody isotyping by ELISA. Serum samples were analyzed for AAV2-specific Ig isotypes (IgG1, IgG2a, and IgG3) by ELISA as described with some modifications (Zhu, J. et al. 2007 J. immunol. 178:3505-3510). Briefly, 96-well plates (Costar) were coated with AAV2-lacZ (1×10⁹ vg/ml) in 100 μl 0.1 M carbonate (pH 9.6) overnight at 4° C. Serial diluted samples were added to antigen-coated plates and incubated for 2 hr. Plates were washed and incubated with biotin-conjugated goat anti-mouse IgG I, IgG2a, and IgG3 (Southern Biotech) for 1 hr. 100 μl of horseradish peroxidase-coupled Streptavidin (BD Biosciences) was then added. Finally, 100 μl per well of the substrate solution (TMB, BD Biosciences) was added and the substrate conversion was stopped by the addition of 100 μl per well of 2 N HCl. Absorbance was measured at 450 nm. Results were expressed as reciprocal endpoint titers as described (Zhu, J. et al. 2007 J. Immunol. 178:3505-3510).

Anti-HA antibody titer. To measure antibody response to HA transgene, HA-expressing Renca cells (Renca-HA), which express HA on cell surface, were seeded on flat 96-well plate. After overnight culture, cells were fixed for 10 min with 0.25% glutaraldehyde in PBS, pH 7.4. Plates were washed and blocked with PBS containing 10% FBS for 2 hr. After washing, serial diluted samples were added and incubated for 2 hr. Plates were then washed and incubated with biotin-conjugated goat anti-mouse IgG (Southern Biotech) for 1 hr at room temperature. Plates were washed as above and TMB substrate solution (BD Biosciences) was added. After 15 min, color development was stopped by the addition of HCl. Optical densities were read at 450 nm on a microplate reader. Results were expressed as reciprocal endpoint titers as described (Zhu, J. et al. 2007 J. Immunol. 178:3505-3510).

Isolation and stimulation of human pDCs. Human pDCs were purified from PBMCs of healthy donors under an institution-sponsored IRB as described (Dzionek, A. et al. 2000 Science 284:1835-1837). Briefly, PBMCs were first depleted of non-pDCs (i.e. T cells, B cells, NK cells, myeloid DCs, monocytes, granulocytes, and erythroid cells) using a cocktail of biotin-conjugated antibodies and anti-biotin microbeads (Miltenyi Biotec). The enriched pDCs were further purified by positive selection using microbeads against human pDC-specific antigen CD304 (BDCA-4/Neuropilin-1) (Miltenyi Biotec). By this two-step magnetic separation procedure, the purity of the isolated pDCs was more than 99%. Monocytes were also purified from PBMCs with anti-CD14 microbeads (Miltenyi Biotec) for use as a control. 5×10⁴ of the purified pDCs and monocytes were stimulated with AAV2-lacZ for 18 hr and cells were harvested for total RNA preparation. The expression of human IFN-α. (hIFN-α) and hIFN-β was assessed by RT-PCR using primers as described (Siegal, F. P. et al. 1999 Science 284:1835-1837): for human IFN-α (sense: 5′-GATGGCCGTGCTGGTGCTCA-3′; antisense: 5′-TGATTTCTGCTCTGACAACCTCCC-3′); and for human IFN-β (sense: 5′-TTGAATGGGAGGCTTGAATA-3; antisense: 5′-CTATGGTCCAGGCACAGTGA-3′). Human ribosomal protein S14 (sense: 5′-GGCAGACCGAGATGAATCCTCA-3′; antisense: 5′-CAGGTCCAGGGGTCTTGGTCC-3′) was used as an internal control. For TLR9 blocking experiments, pDCs were pre-treated with 10 μM of H154 ODN for 30 min, followed by stimulation with AAV2-lacZ or CpG-A ODN. Both H154 ODN (5′-CCTCAAGCTTGAGGGG-3′) and CpG-A ODN (5′-GGGGGACGATCGTCGGGGGG-3′) were phosphorothioate-stabilized, and synthesized by Integrated DNA Technologies.

Statistical analysis. Results are expressed as mean±s.d. Differences between groups were examined for statistical significance using student t-test.

### Examples 13-14

### Blockade of TLR9 and/or type I IFNs Diminishes Adaptive Immune Responses to Adenovirus

### Example 13

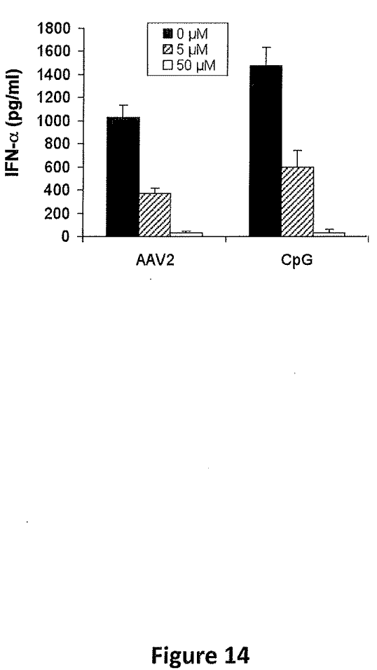

TLR9 blockade diminishes type I IFN production by pDCs. The critical role of TLR9-MyD88 pathway in innate and adaptive immune responses to AAV vectors suggested that blockade of TLR9 may improve the outcome of AAV-mediated gene therapy in vivo. As a first step to test this strategy, purified pDCs were stimulated with AAV2-lacZ in the presence of 0, 5 or 50 μM of TLR9 antagonist, ODN2088 for 18 hrs and the supernatants were assayed for the secretion of IFN-α by ELISA. Our data showed that the addition of TLR9 antagonist significantly reduced the production of IFN-α by AAV-infected pDCs in a dose-dependent manner (FIG. 14). These results indicated that TLR9 blockade was effective in blocking innate immune sensing of AAV in vitro.

### Example 14

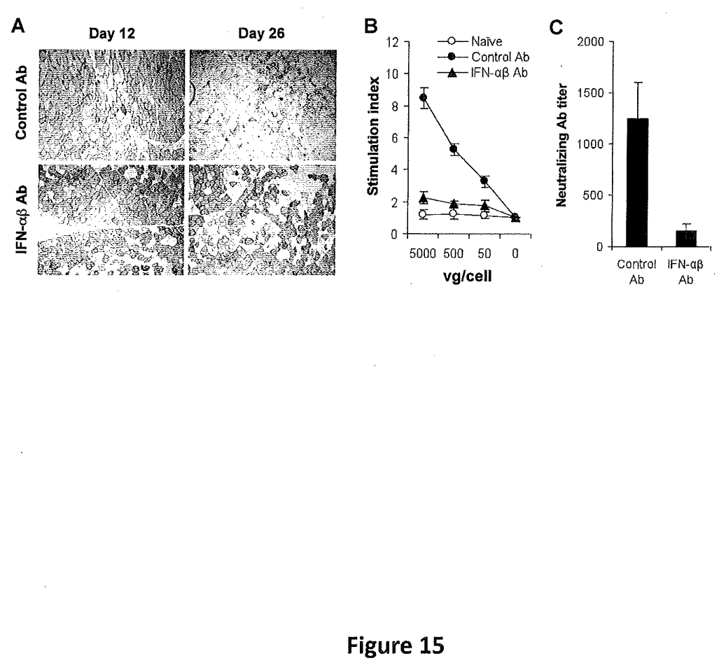

Blockade of Type I IFNs Diminishes Adaptive Immune Responses to Adenovirus. The critical role of type I IFNs in innate and adaptive immune responses to AAV vectors suggested that blockade of type I IFNs may improve the outcome of adenovirus-mediated gene therapy in vivo. To test this strategy, mice were treated with either a control antibody or polyclonal neutralizing antibodies to IFN-α and IFN-β (10,000 IU each; Catalog #31375-3 PBL Biomedical Laboratories) 6 hr prior to infusion of AAV-HA, as well as 3, 6, 9 and 12 days post infusion of AAV-HA. AAV2-HA (1×10¹¹ vg) was injected intramuscularly and examined for HA expression 12 and 26 days after injection. High levels of HA expression were detected in the skeletal muscles of mice 12 days after infection (FIG. 15A). By day 26, significant loss of HA expressing muscle fibers was detected in the control Ab-treated mice. By contrast, the expression of HA remained stable in the IFN-α and IFN-β Abs treated mice (FIG. 15A). This corresponded to a significant (p<0.001) reduction in AAV-specific T cell activation in the IFN-α and IFN-β Abs treated mice compared to the control Ab treated mice (FIG. 15B). In addition, AAV-neutralizing antibody (FIG. 15C) titers were significantly (p<0.001) diminished in the IFN-α and IFN-β Abs treated mice. Taken together, these data indicated that neutralizing antibodies to IFN-α and IFN-β were effective in blocking adaptive immune responses to AAV vectors, leading to improved transgene expression and reduction of adaptive T and B cell responses to AAV in vivo.

Discussion

The adaptive immune responses to AAV represent a significant hurdle in clinical application of AAV vectors for gene therapy. Recent developments have suggested a critical role for the innate immunity in promoting adaptive immune responses. A major unanswered question is how AAV activates the innate immune pathway. In this study, we demonstrated that AAV activated pDCs, but not non-DCs such as cDCs and macrophages, to produce type I IFNs through the TLR9-MyD88 pathway. In vivo, the TLR9-MyD88 innate immune pathway was required for the activation of CD8 T cell responses to both the transgene product and the AAV capsid, leading to the loss of transgene expression. Furthermore, the formation of antibodies to the transgene product and the AAV vector was also critically dependent on this pathway. In addition, we showed that TLR9-dependent activation of adaptive immune responses to AAV was mediated by type I IFNs, and that AAV also activated human pDCs to induce type I IFNs via TLR9.

Our finding that similar to adenoviral vectors, AAV predominantly activated pDCs via the TLR9-MyD88 pathway to secrete type I IFNs is in line with previous observations that pDCs are the most potent type I IFN producers and secrete mainly type I IFNs upon TLR9 stimulation (Zhu, J. et al. (2007) J. Virol. 81:3170-3180; Colonna, M. et al. (2004) Nat. Immunol. 5:1219-1226). The mechanism(s) underlying this pDC-specific involvement of the TLR9-MyD88-type I IFN pathway remains incompletely defined. Studies have shown that stimulation of TLR9 with CpG DNA in pDCs activates MyD88, which then interacts with IRAK1 and TRAF6, leading to the activation of IKKα, and IRF7 and the production of type I IFNs (Hoshino, K. et al. (2006) Nature 440:949-953; Uematsu, S. et al. (2007) J. Biol. Chem. 282:15319-15323). However, this pathway is not operative in non-pDCs such as cDCs. Furthermore, pDCs express high levels of IRF7 and osteopontin, both of which are critical for the induction of type I IFNs (Izaguirre, A. et al. (2003) J. Leukoc. Biol. 74:1125-1138; Shinohara, M. L. et al. (2006) Nat. Immunol. 7:498-506). In addition, CpG DNAs are retained longer in pDC endosomes where TLR9 resides, but are rapidly transferred to lysosomes for degradation in non-pDCs (Honda, K. et al. (2005) Nature 434:1035-1040; Guiducci, C. et al. (2006) J. Exp. Med. 203:1999-2008). Indeed, we have found that pDCs are poorly transduced by adenoviral vectors (Zhu, J. (2007) supra), which may be related to the preferential retention of CpG-containing viral DNA in the endosome.

The TLR9-dependent recognition of AAV also suggests that the ligand for TLR9 recognition is viral DNA. How is the encapsidated single-stranded AAV DNA exposed in endosomes for its recognition by TLR9? It has been shown that following clathrin-dependent or independent internalization, transducing AAV is routed through the endosomal compartment, where pH dependent penetration of endosomes by the virus occurs (Douar, A. M. et al. (2001) J. Virol. 75:1824-1833). Studies with other viruses have suggested that the highly acidified endosomal compartment which contains abundant proteolytic degradation enzymes, may damage viral particles and release some viral DNA for recognition by TLR9 (Kawai, T. et al. (2006) Nat. Immunol. 7:131-137; Crozat, K. et al. (2004) Proc. Natl. Acad. Sci. USA 101835-6836). This process is independent of viral transduction and viruses that do not normally replicate in pDCs, as well as defective viral particles or inactivated virus, can also be detected. Even viruses neutralized by antibody or complement can be taken up via Fc or complement receptors and subject to TLR recognition within endosomes (Wang,. J. P. et al. (2007) J. Immunol. 178:3363-3367). Similar to AAV2, other serotypes of AAV including AAV1 and AAV9 also activate pDCs via the TLR9-MyD88 pathway. However, pDCs infected with AAV1 or AAV9 appear to induce lower levels of type I IFNs than those with AAV2, suggesting different serotypes of AAV may differ in activating the innate immune system. It remains to be defined whether this reflects a difference in endosome targeting and/or processing of AAV. Thus, further investigation is needed to define endosomal sensing of AAV by TLR9.

Whether capsid components of AAV can activate innate immune responses is unknown. A recent report has suggested that complement components might interact with AAV capsid to enhance the stimulation on macrophage by AAV in vitro (Zaiss, A. K. et al. (2008) J. Virol. 82:2727-2740). However, cytokine secretion upon AAV infection is not compromised in mice deficient for complement components in vivo, suggesting that such an interaction may not exist in vivo (Zaiss, A. K. et al. (2008) supra). Thus, the role of complement components in innate immune response to AAV in vivo remains uncertain.

The observation that very low levels of type I IFNs and pro-inflammatory cytokines were produced by non-pDCs upon AAV infection is in striking contrast to adenoviral vectors. Since production of pro-inflammatory cytokines and type I IFNs by pDCs stimulated with adenoviral vectors is mediated by a TLR-independent pathway through cytosolic sensing of double-stranded adenoviral DNA (Zhu, J. et al. (2007) supra; Nociari, M. et al. (2007) J. Virol. 81:4145-4157), these data suggest that the single-stranded AAV genome may not activate this pathway efficiently in non-pDCs. Indeed, it is believed that the ligand for the yet-to-be-identified cytosolic DNA sensor is double stranded B-form DNA derived from many microbes (Stetson, D. B. et al. (2006) Immunity 24:93-103; Ishii, K. J. et al. (2006) Nat. Immunol. 7:40-48). The very low levels of cytokines produced by non-pDCs upon AAV infection may explain a lack of strong inflammatory responses documented in numerous models of AAV-mediated gene therapy in vivo (Zaiss, A. K. et al. (2005) Curr. Gene Ther. 5:323-331). As pDCs mainly reside in the spleen and other secondary lymphoid organs (Colonna, M. et al. (2004) Nat. Immunol. 5:1219-1226), the lack of strong type I IFN or pro-inflammatory cytokine responses from non-pDCs upon AAV infection may also explain a recent observation that robust transcriptome responses, including the induction of a cluster of type I IFN-related genes, associated with adenoviral vectors by microarray analysis of the liver RNA were not observed with AAV vectors (McCaffrey, A. P. et al. (2008) Mol. Ther. 16:931-941). Taken together, the above observations are consistent with the notion that AAV is a weak immunogen compared to adenoviral vectors.

The biological significance of the TLR9-MyD88-type I IFN pathway in innate sensing of AAV by pDCs lies in its critical role in the activation of adaptive T and B cell responses to the transgene product and the AAV vector. Our results indicate that an intact TLR9-MyD88 pathway is required for the activation of both AAV capsid- and transgene product-specific CD8 T cells. The lack of CD8 T cell responses early after infection (day 12) in TLR9^{-/-} or MyD88^{-/-} mice suggests that the TLR9-MyD88 pathway is critical for CD8 T cell priming. The observation that the kinetics of transgene-specific CD8 T cell response is closely associated with the loss of transgene expression (FIG. 6A, E, F), suggests that transgene-specific CD8 T cells may be critical for the elimination of the transduced cells in vivo. This is very similar to a recent observation that lentiviral vectors activate pDCs via TLR7 to secrete type IFNs, which is required for subsequent activation of CTLs against the transgene product (Brown, B. D. et al. (2007) Blood 109:2797-2805). However, since the TLR9 pathway also regulates the activation of capsid-specific CD8 T cells, we cannot rule out the role of capsid-specific T cells in eliminating AAV-transduced cells in vivo.

The mechanism(s) underlying type I IFN-dependent adaptive immune responses to AAV requires further investigation. Studies in other models have shown that type I IFNs can promote DC maturation and function (Honda, K. et al. (2003) Proc. Natl. Acad. Sci. USA 100:10872-10877; Hoebe, K. et al. (2003) Nat. Immunol. 4:1223-1229). Type I IFNs have also been shown to enhance cross-presentation by DCs (Le Bon, A. et al. (2003) Nat. Immunol. 4:1009-1015). This observation is particularly relevant to AAV infection since the activation of both transgene- and viral capsid-specific CTL responses are thought to be dependent on cross-presentation by MHC class I (Manning, W. C. et al. (1997) J. Virol. 71:7960-7962; Sarukhan, A. et al. (2001) J. Virol. 7569-277; Vandenberghe, L. H. et al. (2006) Nat. Med. 12:967-971; Wang, Z. et al. (2007) Hum. Gene Ther. 18:185-194; Li, C. et al. (2007) J. Virol. 81:7540-7547). Furthermore, we have recently shown that direct type I IFN signaling is required for the survival of activated T cells in response to vaccinia viral infection (Quigley, M. et al. (2008) J. Immunol. 180:2158-2164). Type I IFNs are also critical for multiple stages of adaptive B cell response to adenovirus, and the generation of protective neutralizing antibodies to adenovirus critically depends on type I IFN signaling on both CD4 T cells and B cells (Zhu, J. et al. (2007) J. Immunol. 178:3505-3510). Thus, future studies should focus on defining mechanisms by which type I IFNs promote adaptive immune responses to AAV.

Our observation that human pDCs can also be activated by AAV to induce type I IFNs via TLR9, suggests that the TLR9 pathway might also be important in regulating adaptive immune responses to AAV in humans. However, studies have shown that the induction of adaptive immune responses to AAV is influenced by many factors including host species and the pre-existing immunity to AAV (Vandenberghe, L. H. et al. (2007) Curr. Gene Ther. 725-333). Thus, additional studies in non-human primates as well as in human clinical trials are required to define the role of TLR9 innate immune pathway in the activation of adaptive immune responses to AAV in humans.

The route of administration has also played a role in adaptive immune responses to AAV. Studies in murine models have shown that hepatic delivery of AAV often results in immune tolerance to the transgene product (Ge, Y. et al. 2001) Blood 97:3733-3737; Xiao, W. et al. (2000) Mol. Ther. 1:323-329). It is not clear whether this is a result of defective innate immune activation in the liver (e.g., insufficient pDCs or lack of interaction of AAV with pDCs), or the existence of immunosuppressive cell types such as regulatory T cells and Kupffer cells, and/or immunosuppressive cytokines such as IL-10 in the hepatic microenvironment (Erhardt, A. et al. (2007) Hepatology 45:475-485; You, Q. et al. Hepatology 48:978-990). Thus, it will be important to delineate factors that influence immune responses to AAV in the liver.

In conclusion, our study reveals that AAV activates the innate immunity through the TLR9-MyD88 pathway in pDCs, which leads to the production of type I IFNs. In vivo, this innate immune pathway plays a critical role in the activation of CD8 T cell responses to both the transgene product and the AAV capsid, and the formation of anti-transgene and AAV-neutralizing antibodies. Furthermore, AAV also activates human pDCs to produce type I IFNs in a TLR9-dependent fashion. These results suggest that strategies targeted to interfere with the TLR9-MyD88-type I IFN signaling pathway may improve the safety and efficacy of AAV vectors for gene therapy in humans.

Materials and Methods

Mice. C57BL/6 and BALB/c mice were purchased from the Jackson Laboratory. TLR2^{-/-}, TLR9^{-/-}, MyD88^{-/-}, and TRIF^{-/-} mice on C57BL/6 background were kindly provided by Shizuo Akira (Osaka University, Osaka, Japan). IFN-αβR^{-/-} mice (Muller, U. et al. (1994) Science 264:1918-1921) on 129/Sv background and their normal control 129/Sv mice were obtained from B & K Universal. TLR9^{-/-} and MyD88^{-/-} mice have been backcrossed onto BALB/c background for more than nine generations in our animal facility. Groups of 7˜10 wk-old mice were selected for this study. All experiments involving the use of mice were done in accordance with protocols approved by the Animal Care and Use Committee of Duke University.

Recombinant AAV. Recombinant AAV2 encoding influenza hemagglutinin (AAV2-HA), lac Z (AAV2-lacZ) or GFP (AAV2-GFP) under the control of CMV promoter were generated with a helper virus-free system (Stratagene) by transfecting 293 cells (which stably express the adenovirus E1 gene) with three plasmids as described Matsushita, T. et al. (1998) Gene Ther. 5:938-945; Xiao, X. et al. (1998) J. Virol. 72:2224-2232). These three plasmids are pHelper, encoding E2A, E4 and VA RNA genes of adenovirus; pAAV-RC, encoding AAV2 rep and cap genes; and vector plasmid pAAV-CMV-HA, lacZ or GFP. Purification of AAV was done by heparin affinity chromatography as described (Auricchio, A. et al. (2001) Hum. Gene Ther. 12:71-76). Briefly, cells were disrupted by freezing and thawing two times and cell lysates were incubated with 40 μg/ml of DNase 1 and RNase A (Roche Biochemicals) for 30 min at 37° C. After centrifugation, the supernatants were then incubated with 0.5% deoxycholic acid (Sigma) for 30 min at 37° C., followed by filtration through a 5-μm and a 0.8-μm pore size filter (Millipore) sequentially. The cleared supernatants were then loaded onto a heparin column. After washing twice with PBS, pH7.4, plus 0.1 M NaCl, the virus was eluted with PBS, pH7.4, plus 0.4 M NaCl. The eluate was concentrated with a Millipore Biomax-100K NMWL filter device by centrifugation. The viral genome (vg) titer was determined by a CMV promoter-specific quantitative real time PCR procedure.

For the production of recombinant AAV1-GFP and AAV9-GFP, pAAV-CMV-GFP and pHelper were co-transfected into 293 cells along with a chimeric packaging plasmid in which the AAV2 rep gene was fused to AAV1 and AAV9 capsid genes, respectively. Cells were harvested, sonicated and treated with DNase 1 and RNase A. The resultant AAV1-GFP and AAV9-GFP viral particles were purified twice by CsCl density gradient ultracentrifugation as described (Auriccho, A. et al. (2002) J. Clin. Invest. 110:499-504).

Murine DC culture. pDCs were generated as described (Zhu, J. et al. (2007) supra). Briefly, bone marrow cells were harvested from femurs and tibiae of mice and cultured in the presence of 200 ng/ml of Flt-3 ligand (R & D Systems) for 9 days. For generation of cDCs, bone marrow cells were in the presence of mouse GM-CSF (1,000 U/ml) and IL-4 (500 U/ml) (R & D Systems) for 5 days as described (Yang, Y. et al. (2004) Nat. Immunol. 5:508-515). pDCs and cDCs were stained with anti-B220-FITC and anti-CD11c-PE and purified by FACS sorting. Purified cells were then stimulated with various agents at a density of 1×10⁶ cells/ml.

Isolation of murine splenic DCs, macrophages and Kupffer cells. Splenic DC isolation was performed as described (Zhu, J. et al. (2007) supra). After perfusion with Liberase CI (Roche Biochemicals), single cell suspensions were subjected to 30% BSA gradient, and the interface DC fraction was collected and stained with anti-B220-FITC and anti-CD11c-biotin followed by streptavidin-microbeads (Miltenyi Biotec). CD11c⁺ DCs were purified by positive selection by microbeads and subjected to FACS sorting into pDCs (CD11c⁺B220⁺) and cDCs (CD11c⁺B220⁻). Macrophages were isolated from the peritoneal cavity of mice 3 days after intraperitoneal injection of 2.5 ml of 3% thioglycollate as described (Lund, J. M. et al. (2004) Proc. Natl. Acad. Sci. USA 101:5598-5603), Kupffer cells were isolated from mouse livers as described (31). After perfusion in situ via portal vein with collagenase, single cell suspensions were subjected to gradient centrifugation with 11.5% OptiPrep solution. Kupffer cell fraction was collected from the interface and purified by FACS sorting for F4/80 positive cells. Purified splenic CD1 DCs, macrophages and Kupffer cells were stimulated with various agents at a density of 2.5×10⁵ cells/ml.

Detection of cytokines by ELISA. Production of IL-6, TNF-α, IFN-α and IFN-β by DCs in response to various stimuli was detected in culture supernatants by ELISA kits according to manufacturer's standard protocols. IL-6 and TNF-α ELISA kits were purchased from Endogen Pierce. IFN-α and IFN-β kits were obtained from PBL Biomedical Laboratories.

In vivo delivery of recombinant AAV 1×10¹¹ vg of AAV-HA in 25 μl was injected into tibialis anterior muscles of mice. Mice were sacrificed at indicated time points for histological and immunological assays. All animals that received recombinant virus survived to the time of necropsy.

Proliferation assay. T cells were isolated from splenocytes using CD5-microbeads (Miltenyi Biotec). CD5⁺ T cells (2×10⁵) were co-cultured with irradiated (3000 rad) naïve splenocytes (2×10⁵) in the presence of AAV2-HA at 0, 50, 500, or 5000 vg/cell for 72 hr in triplicates. Cultures were pulsed with 1 μCi per well of ³H-thymidine. 16-20 hr after pulsing, plates were harvested using a 96-well cell harvester and the ³H-thymidine incorporation was counted using a 1205 Betaplate scintillation counter (Wallace).

Antibodies and flow cytometry. All antibodies used for FACS were purchased from BD Biosciences. FACSCanto (BD Biosciences) was used for flow cytometry event collection and data were analyzed using FACS DNA and CELLQuest software (BD Biosciences).

For intracellular IFN-γ staining, splenocytes were stimulated with 2 μg/ml of L^{d}-restricted AAV-2 capsid epitope peptide)(³⁷²VPQYGYLTL³⁸⁰) (Sabatino, D. E. et al. (2005) Mol. Ther. 12:1023-1033) or K^{d}-restricted HA epitope peptide (⁵¹⁸¹YSTVASSL⁵²⁶) (Yang, Y. et al. (2004) supra), and 5 μg/ml of GolgiPlug (BD Biosciences) for 5 hr. After washing, cells were stained with anti-CD8 (Clone 53-6.7) and permeabilized to detect IFN-γ intracellularly with anti-IFN-γ (Clone XMG1.2) using the Cytofix/Cytoperm kit (BD Biosciences) as previously described (Yang, Y. et al. (2004) supra).

Immunohistochemical staining. Frozen sections (5 μm) of muscles was fixed with acetone, air dried and rehydrated in PBS. After blocking with 20% goat-serum in PBS, sections were stained with biotinylated anti-HA or anti-CD8 mAb, followed by ABC kit (Vector Laboratories) as described (Huang, X. et al. (2004) Eur. J. Immunol. 34:1351-1360).

Neutralizing antibody assay. Neutralizing antibody titers were analyzed by assessing the ability of serum antibody to inhibit transduction of AAV2-LacZ into AAV permissive HT1080 cell line. 60-70% confluent HT1080 cells in 96-well plates (2×10⁴ cells per well) were treated with 0.2 ml of 240 mM of hydroxyurea and 3 mM of sodium butyrate for 6 hr at 37° C. Serum samples were incubated at 56° C. for 30 mM and then diluted in 2-fold steps starting from 1:20 or 1:50. Serial dilutions of sera were pre-incubated with 2.5×10⁸ vg of AAV2-lacZ in a 100 μl total volume for 1 hr at 37° C., and added to pre-treated cell cultures. Cells were fixed and analyzed for lacZ expression by X-gal staining on the following day as described (Zhu, J. et al. (2007) supra). All of the cells stained blue in the absence of serum samples. The titer of neutralizing antibody for each sample was reported as the highest dilution with which less than 50% of cells stained blue.

AAV2-specific antibody isotyping by ELISA. Serum samples were analyzed for AAV2-specific Ig isotypes (IgG1, IgG2a, and IgG3) by ELISA as described with some modifications (Zhu, J. et al. (2007) supra). Briefly, 96-well plates (Costar) were coated with AAV2-lacZ (1×10⁹ vg/ml) in 100 μl 0.1 M carbonate (pH 9.6) overnight at 4° C. Serial diluted samples were added to antigen-coated plates and incubated for 2 hr. Plates were washed and incubated with biotin-conjugated goat anti-mouse IgG1, IgG2a, and IgG3 (Southern Biotech) for 1 hr. 100 μl of horseradish peroxidase-coupled Streptavidin (BD Biosciences) was then added. Finally, 100 μl per well of the substrate solution (TMB, BD Biosciences) was added and the substrate conversion was stopped by the addition of 100 μl per well of 2 N HCl. Absorbance was measured at 450 nm. Results were expressed as reciprocal endpoint titers as described (Zhu, J. et al. (2007) supra).

Anti-HA antibody titer. To measure antibody response to HA transgene, HA-expressing Renca cells (Renca-HA), which express HA on cell surface, were seeded on flat 96-well plate. After overnight culture, cells were fixed for 10 min with 0.25% glutaraldehyde in PBS, pH 7.4. Plates were washed and blocked with PBS containing 10% FBS for 2 hr. After washing, serial diluted samples were added and incubated for 2 hr. Plates were then washed and incubated with biotin-conjugated goat anti-mouse IgG (Southern Biotech) for 1 hr at room temperature. Plates were washed as above and TMB substrate solution (BD Biosciences) was added. After 15 min, color development was stopped by the addition of HCl. Optical densities were read at 450 nm on a microplate reader. Results were expressed as reciprocal endpoint titers as described (Zhu, J. et al. (2007) supra).

Isolation and stimulation of human pDCs. Human pDCs were purified from PBMCs of healthy donors under an institution-sponsored IRB as described (69). Briefly, PBMCs were first depleted of non-pDCs (i.e. T cells, B cells, NK cells, myeloid DCs, monocytes, granulocytes, and erythroid cells) using a cocktail of biotin-conjugated antibodies and anti-biotin microbeads (Miltenyi Biotec). The enriched pDCs were further purified by positive selection using microbeads against human pDC-specific antigen CD304 (BDCA-4/Neuropilin-1) (Miltenyi Biotec). By this two-step magnetic separation procedure, the purity of the isolated pDCs was more than 99%. Monocytes were also purified from PBMCs with anti-CD14 microbeads (Miltenyi Biotec) for use as a control. 5×10⁴ of the purified pDCs and monocytes were stimulated with AAV2-lacZ for 18 hr and cells were harvested for total RNA preparation. The expression of human IFN-α (hIFN-α) and hIFN-β was assessed by RT-PCR using primers as described (70): for human IFN-α (sense: 5′-GATGGCCGTGCTGGTGCTCA-3′; antisense: 5′-TGATTTCTGCTCTGACAACCTCCC-3′); and for human IFN-β (sense: 5′-TTGAATGGGAGGCTTGAATA-3; antisense: 5′-CTATGGTCCAGGCACAGT GA-3′). Human ribosomal protein S14 (sense: 5′-GGCAGACCGAGATGAATCCTCA-3′; antisense: 5′-CAGGTCCAGGGGTCTTGGTCC-3′) was used as an internal control. For TLR9 blocking experiments, pDCs were pre-treated with 10 μM of H154 ODN for 30 min, followed by stimulation with AAV2-lacZ or CpG-A ODN. Both H154 ODN (5′-CCTCAAGCTTGAGGGG-3′) and CpG-A ODN (5′-GGGGGACGATCGTCGGGGGG-3′) were phosphorothioate-stabilized, and synthesized by Integrated DNA Technologies.

Statistical analysis. Results are expressed as mean±s.d. Differences between groups were examined for statistical significance using student t-test.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to early out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

We claim:

1. A method of inhibiting in a subject formation of neutralizing antibodies directed against a recombinant viral vector comprising co-administering to said subject said viral vector and an interfering molecule, wherein said interfering molecule is capable of disrupting the TLR9-MyD88-type I IFN signaling pathway.

2. The method according to claim 1, further comprising the step of re-administering said viral vector to said subject.

3. The method according to claim 1, wherein said interfering molecule is administered simultaneously with said viral vector.

4. The method according to claim 1, wherein said interfering molecule is administered prior to said administration of said viral vector.

5. The method according to claim 1, wherein said interfering molecule is administered subsequently after the administration of said viral vector.

6. The method according to claim 1, wherein said interfering molecule is selected from the group consisting of an antagonist, antisense RNA, siRNA, aptamers, and combinations thereof.

7. The method according to claim 6, wherein said interfering molecule comprises an antagonist.

8. The method according to claim 7, wherein said antagonist comprises H154ODN.

9. The method according to claim 7, wherein said antagonist comprises ODN2088.

10. The method according to claim 1, wherein said viral vector comprises an adeno-associated virus (AAV).

11. A method of inhibiting in a subject formation of an immune response directed against a viral vector comprising co-administering to said subject said viral vector and an interfering molecule directed against type I interferons, wherein the formation of said immune response is inhibited.

12. The method according to claim 11, further comprising the step of re-administering said viral vector to said subject.

13. The method according to claim 11, wherein said interfering molecule is administered simultaneously with said viral vector.

14. The method according to claim 11, wherein said interfering molecule is administered prior to said administration of said viral vector.

15. The method according to claim 11, wherein said interfering molecule is administered subsequently after the administration of said viral vector.

16. The method according to claim 11, wherein said interfering molecule comprises a polyclonal neutralizing antibody directed to INF-α or IFN-β.

17. The method according to claim 11, wherein said viral vector comprises an adeno-associated virus (AAV).


SEQUENCE LISTING (10 sequences)

SEQ ID NO: 1 - protein, 9 aa, adeno-associated virus 2
VPQYGYLTL

SEQ ID NO: 2 - protein, 9 aa, Homo sapiens
IYSTVASSL

SEQ ID NO: 3 - DNA, 20 bp, Homo sapiens
gatggccgtgctggtgctca

SEQ ID NO: 4 - DNA, 24 bp, Homo sapiens
tgatttctgctctgacaacctccc

SEQ ID NO: 5 - DNA, 20 bp, Homo sapiens
ttgaatgggaggcttgaata

SEQ ID NO: 6 - DNA, 20 bp, Homo sapiens
ctatggtccaggcacagtga

SEQ ID NO: 7 - DNA, 22 bp, Homo sapiens
ggcagaccgagatgaatcctca

SEQ ID NO: 8 - DNA, 21 bp, Homo sapiens
caggtccaggggtcttggtcc

SEQ ID NO: 9 - DNA, 16 bp, Homo sapiens
cctcaagcttgagggg

SEQ ID NO: 10 - DNA, 20 bp, Homo sapiens
gggggacgatcgtcgggggg